(19) United States
(12) Patent Application Publication
(10) Pub. No.: US 2016/0120949 A1
(43) Pub. Date: May 5, 2016

(54) USES OF IL-12 IN HEMATOPOIETIC IMMUNOTHERAPY (HIT)

(71) Applicant: Neumedicines, Inc., Pasadena, CA (US)

(72) Inventor: Lena A. BASILE, Tujunga, CA (US)

(73) Assignee: NEUMEDICINES, INC., Pasadena, CA (US)

(21) Appl. No.: 14/928,499

(22) Filed: Oct. 30, 2015

Related U.S. Application Data
(60) Provisional application No. 62/073,220, filed on Oct. 31, 2014.

(51) Int. Cl. A61K 38/20

(52) U.S. Cl. CPC A61K 38/208

(57) ABSTRACT

Aspects and embodiments of the instant disclosure provide therapeutic methods and compositions comprising interleukin 12 (IL-12) as a hematopoietic immunotherapy (HIT) useful for treating or preventing a cancer patient from chemotherapy-induced cytopenias necessitating a dose reduction and/or dose delay. following exposure of the patient to chemotherapeutic agents, the method comprising: administering a dose of therapeutically effective amount of a pharmaceutical composition comprising substantially isolated IL-12 to the subject, whereby cytopenias are reduced and leading to increases responses to the chemotherapy agent(s).diminished.

### CROSS-REFERENCE TO RELATED APPLICATIONS

This application claims the benefit of priority from U.S. Provisional Patent Application No. 62/073,220, filed Oct. 31, 2014. The entire contents of which are incorporated herein by reference in its entirety.

### FIELD

The present disclosure relates generally to novel methods and compositions for transplantation. In particular, methods and compositions for use in hematopoietic transplantation comprising administering to a subject in need thereof a therapeutically effective amount of a pharmaceutical composition comprising IL-12. The use of IL-12 in the invention tackles two problems in the use of chemotherapy for the treatment of cancer: 1) cytopenias caused by chemotherapeutic regimens, leading to dose reductions and/or dose delays and other complications are reduced, and 2) reduction of the underlying cancer. Both aspects of the invention result in an increase in response rates due to the chemotherapy, including reduction of minimal residual disease (MRD), thereby leading to longer survival times for the treated patients. Because of these dual effects of IL-12 in cancer patients requiring treatment with chemotherapy, we refer to IL-12 as a hematopoietic immunotherapy (HIT).

### BACKGROUND

The following includes information that may be useful in understanding various aspects and embodiments of the present disclosure. It is not an admission that any of the information provided herein is prior art, or relevant, to the presently described or claimed inventions, or that any publication or document that is specifically or implicitly referenced is prior art.

Hematopoietic cytopenias including chemotherapy induced thrombocytopenia (CIT), are a common side effect of the bone marrow suppression caused by chemotherapy treatment. Platinum-containing regimens are especially correlated with incidences of Grade 3 and 4 thrombocytopenia, as well as Grade 3 and 4 neutropenia, lymphopenia and anemia. Risks associated with CIT include increased incidence of hemorrhage; chemo treatment dose reductions and schedule alterations; and an increased need for platelet transfusions. Of the 1.4 million patients who receive chemotherapy annually in the United States, approximately 10% suffer from thrombocytopenia (MGI press release Aug. 28, 2007). Overall annual incidences of CIT are expected to continue to increase because new, more aggressive chemotherapy agents are entering the market and standard of care treatments continue to change and evolve.

Currently, the complications associated with CIT reach a level of incidence of approximately 60%. Thrombocytopenia can lead to bruises with no provocation. Platelet counts less than 50,000/ul are associated with a high risk of spontaneous bleeding. Platelets also carry the sleep, mood and appetite regulating neurotransmitter, serotonin, and its precursor, l-tryptophan, therefore, thrombocytopenia is often accompanied by fatigue and sometimes depression. Thrombocytopenia also can impede a variety of diagnostic and/or medical treatments. Further, platelets are now known to have immune modulating effects (Semple and Freedman Cell Mol Life Sci 67:499 2010). Although thrombocytopenia is generally associated with bleeding and hemorrhage, a lesser-known problem is the effect of low platelets on the immune system. Platelets have been found to be capable of maturing dendritic cells, which are key cells of the innate and adaptive immune systems [Hamzeh-Cognasse et al BMC Immunol 9:54 2008]. Still other recent reports show that thrombocytopenia is linked to increased risk of death due to disease relapse following transplantation in cancer patients [Ninan et al Biol Blood Marrow Transpl 13:895 2007].

There are no drugs available for use for the prevention or treatment of CIT. In 1997, Interleukin-11 (IL-11, oprelvekin) was approved by the US FDA for the prevention of severe thrombocytopenia and the reduction of the need for platelet transfusions following myelosuppressive chemotherapy in adult patients with nonmyeloid malignancies who are at high risk for severe thrombocytopenia. However, due to the serious side effects of the drug, as well as its limited efficacy, IL-11 is generally not used in clinical practice in oncology. Physicians are concerned about the ocular and cardiovascular side effects of the drug and are unwilling to administer it to the majority of patients. Instead thrombocytopenia is managed by chemotherapy dose reductions, dose delays, and, when severe, platelet transfusions. Of further significance is the fact that newer thrombopoietic drugs recently approved for immune thrombocytopenic purpura (ITP), Nplate (Romiplostim) and Promacta (eltrombopag), have not been successful in meeting clinical endpoints in Phase II study.

### SUMMARY OF THE INVENTION

Accordingly, there is a need for novel methods and compositions useful for effective treatment or prevention of CIT and other cytopenias, and at the same time provide anti-tumor responses that can combat the underlying cancer (reduce tumor burden and reduce MRD), leading to longer survival times.

The present disclosure provides therapeutic methods and compositions comprising interleukin 12 (IL-12) useful for treating or preventing a subject, who has cancer and will receive chemotherapy prior to transplantation, from various cytopenias, particularly thrombocytopenia following exposure of the subject to chemotherapeutic agents. In some aspects the method comprises: administering a dose of therapeutically effective amount of a pharmaceutical composition comprising substantially isolated IL-12 to the subject susceptible to or having following exposure of the subject to chemotherapeutic agents, whereby cytopenias are prevented, diminished and/or treated. Along with the effects of IL-12 on multiple cytopenias, a concomitant effect will be on the reduction of tumor burden and/or a reduction MRD. Overall these dual effects lead to longer survival in cancer patients in need of treatment with chemotherapy.

In one embodiment, the method comprises administering one or more doses of the chemotherapeutic agent according to the need of the subject.

In one embodiment, the method comprises administering one or more doses of IL-12 before, during and/or after the administration of the chemotherapeutic agent according to the need of the subject and/or to prevent, diminish and/or treat cytopenias.

In one embodiment, the method comprises administering various types of chemotherapeutic agent according to the need of the subject.

In one aspect, the one or more effective doses of IL-12 are administered subcutaneously, intravenously, intraperitoneally, intramuscularly, epidurally, parenterally.

In one aspect, the administered IL-12 induces the production of platelets, lymphocytes, including natural killer cells and CD8 cells, red blood cells and neutrophils in peripheral blood. In some aspects, the platelet and red blood cell production enhances hematopoeitic regeneration or reconstitution and/or survival, and/or decreasing the need for platelet or red blood cell (or whole blood) transfusions, the risk of bleeding and/or hemorrhage. Increases in white blood cells, neutrophils and lymphocytes, lead to a decrease in infectious complications.

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction. Additional embodiments may be disclosed in the Detailed Description below.

### BRIEF DESCRIPTION OF THE FIGURES

FIGS. 1A-1B: Blood Recovery Profiles for Vehicle and rMuIL-12 Administered Before and After Myelosuppresive Radiation (625 rad). Blood cell recovery profiles are shown for the EL4 lymphoma tumor model (A) and the Lewis lung cancer model (B) for neutrophils, red blood cells and platelets. Pre-radiation blood values reflect an average of all animals in both groups before radiation and tumor inoculation. The normal threshold value for murine blood cell counts is indicated by the dashed line. During days 14-21 in both tumor models, rMuIL-12-treated mice showed statistically significant improvements in blood cell counts as compared to the vehicle control (P<0.001 for days 14 and 18 and P<0.01 at day 21; ANOVA followed by Tukey tests).

FIGS. 2A-2B: Relative Changes in Tumor Volumes for Vehicle and rMuIL-12 Treatment Groups Following Radiation (625 rad). Changes in tumor volumes over the course of the experiment are shown for the EL4 lymphoma tumor model (A) and the Lewis Lung cancer tumor model (B). Mice in both tumor models were given 625 rad on day 1. Mice in both tumor models were given a second dose of radiation on day 22 following the initial radiation dose. In the EL4 lymphoma model, all rMuIL-12 treatment groups, namely pre, post and pre-post radiation dosing groups, significantly reduced tumor growth (% T/C<50%) as compared with the control at the endpoint of tumor volume evaluation. In the Lewis lung cancer model, rMuIL-12 post-treatment significantly reduced tumor growth (% T/C<50%) at the endpoint of tumor growth evaluation.

FIG. 3: Kaplan-Meier Survival Curves of Irradiated, Unsupported Monkeys Treated with HemaMax™. Pooled HemaMax™ dosing group is shown. No antibiotics were used during the study. One animal with a broken tooth was excluded from the study because of an unrelated death to the study. p<0.05 pooled treatment groups.

FIGS. 4A-4B: Leukocyte (A) and Thrombocyte (B) Counts in Irradiated, Unsupported Rhesus Monkeys Treated With HemaMax™. Three analyses were conducted to assess differences in blood cell counts during the study period. In the first analysis, where blood cell counts were analyzed from day 1 up to day 30, animals treated with HemaMax™ had significantly higher numbers of leukocytes and thrombocytes at days 12 and 14 for the 100 ng/kg and 250 ng/kg doses of HemaMax™ as compared to animals treated with vehicle. Notably, in this study lethal radiation NHP study, 80% of vehicle-treated monkeys required a platelet transfusion, whereas only 25% of HemaMax-treated monkey required a platelet transfusion (p<0.007, chi square analysis).

FIGS. 5A-5B: Human and NHP Bone Marrow Express IL-12Rβ2. Tissues from human (A) and NHP (B) femoral bone marrow were immunohistochemically stained for IL-12Rβ2. Progenitor cells and megakaryocytes expressing IL-12Rβ2 are shown. Plos One paper FIG. 10a.

FIGS. 6A-6F: Murine IL-12 Promotes Hematopoietic Recovery in Irradiated Mice. Representative sections of femoral bone marrow from non-irradiated, untreated mice that were stained for IL-12Rβ2 are shown in (A). Animals were subjected to TBI (8.0 Gy) and subsequently received vehicle or rMuIL-12 (20 ng/mouse) subcutaneously at the indicated times post irradiation (B-F). Femoral bone marrow was immunohistochemically stained for IL-12Rβ2 (orange color) 12 days after irradiation. While bone marrow from mice treated with vehicle lacked IL-12Rβ2-expressing cells and showed no signs of hematopoietic regeneration (B), mice treated with rMuIL-12 showed hematopoietic reconstitution and the presence of IL-12Rβ2-expressing megakaryocytes, myeloid progenitors, and osteoblasts (C-F). Magnification=100×.

FIG. 7: Lin-IL-12Rβ2-cells were plated in megacult medium at 5000 cells/slide with and without IL-12. Cultures stimulated with IL-12 resulted in larger colonies in comparison to cultures with media alone.

FIG. 8: Percentage of Individual Platelet Counts Compared to Baseline in CIT Mice Treated with rMuIL-12. None of the rMuIL-12 treated groups had an animal with a decrease in platelets of more than 33% (0 out of 50). In contrast the control group which did not receive rMuIL-12 had 6 mice out of 20 (30%) in which platelet levels compared to basal dropped to 33% or less.

FIG. 9: Lewis Lung Tumor Volume of Mice Under Therapy with GC Receiving rMuIL-12. Note: Time zero represents the time of chemotherapy administration. Tumor inoculation took place 11 days prior to chemotherapy administration.

FIG. 10. Lewis Lung Tumor Volume of Mice Under Monotherapy With Gemcitabine Receiving rMuIL-12. Day 0 is the time of the first chemotherapy. The second chemotherapy administration took place at day 7. Tumor inoculation took place on day 8. rMuIL-12 did not increase the tumor growth rate in this model system using only gemcitabine as the chemotherapy agent. The differences in growth among groups were not statistically significant. With the exception of the pre-dosing group, a trend for decreased tumor size was observed with rMuIL-12 administration.

### DETAILED DESCRIPTION

Cytotoxic chemotherapy has proven useful in the treatment of a myriad of malignant diseases, but is associated with myelosuppression of varying degrees. Neutropenia, anemia, and thrombocytopenia frequently are dose-limiting toxicities that may limit timely and effective dosing of chemotherapy. Commercially available hematopoietic growth factors have been used very effectively both for the treatment/prevention of neutropenia (Neupogen, Neulasta, Leukine), and also to treat chemotherapy-induced anemia (Procrit, Aranesp). The only available treatment for chemotherapy-induced thrombocytopenia (Neumega) has not been widely used due to untoward side effects. Prevention of chemotherapy-induced cytopenias allows for maintaining dose intensity of chemotherapy, which may in turn ultimately lead to better patient outcomes.

Platelet Transfusions: With lack of available drugs, the only available treatment for acute or chronic thrombocytopenia is platelet transfusions. Approximately 2 million platelet transfusions are given each year in the US (Heala and Blumberg Blood Reviews 18:149 2004). Transfusions only temporarily correct thrombocytopenia and are associated with high rates of complications (about 60%). Platelet transfusions can have serious side effects. Patients receiving platelet transfusions are at risk for several reactions that range from mild allergic reactions to life-threatening anaphylaxis. Febrile reactions are the most common, occurring in 1 in every 100 transfusions. Clinically, the most significant complications are the immunomodulatory effects of alloimmunization, immunosuppression and graft-versus-host disease (GVHD). Also patients receiving platelet transfusions are at risk for bacterial, parasitic and viral infections. Patients receiving an allogeneic transfusion are at greater risk for lethal infection for the hepatitis viruses than from HIV.

Accordingly, the instant disclosure is directed to HemaMax™ (IL-12) as an adjunctive treatment in combination with chemotherapy to treat/prevent chemotherapy-induced thrombocytopenia (CIT) and other cytopenias, and also provide anti-tumor responses that result in a reduction in MRD. Together these two effects lead to cancer patients have longer progression free survival and overall survival times.

HemaMax™ (rHuIL-12): is a heterodimeric protein consisting of two subunits linked by disulfide bonds. The two subunits are an A and B subunit referred to as p35 and p40, respectively. Heterodimeric IL-12 contains 503 amino acids. The protein can be produced by the recombinant protein production technology in Chines Hamster Ovary (CHO) cells with a total molecular weight of about 75.0 kDa and, like endogenous IL-12, is a glycoprotein in its final form. The glycosylation pattern of HemaMax™ is different from endogenous IL-12. HemaMax™ potently elicits the pharmacodynamic response (interferon-γ [IFN-γ]) in human immune cells in vitro and in non-human primates (rhesus monkeys) both in vitro and in vivo.

HemaMax (rHuIL-12): HemaMax has demonstrated excellent blood cell recovery, including platelet recovery, recovery following myelosuppressive or myeloablative therapies in murine models, as well as in a non-human primate (NHP) model following myeloablative treatment (see Section D). In fact, in our proof-of-concept, lethal radiation NHP study, 80% of vehicle-treated monkeys required a platelet transfusion, whereas only 25% of HemaMax-treated monkey required a platelet transfusion (p<0.007, chi square analysis). These results present strong evidence in support of advancing HemaMax in CIT. HemaMax's mechanism of action (MOA) involves regenerating hematopoiesis at the level of hematopoietic stem cells (HSC). In support of this MOA, Neumedicines has found the IL-12 receptor on several key subpopulations of human HSC, as it is co-expressed along with known stem cell markers, such as CD34, c-Kit and KDR. Further, going down the line in differentiation, the IL-12 receptor has been found on both human and murine megakaryocytes both immature and mature megakaryocytes, and even on platelets themselves. Thus, the MOA of HemaMax portends success in meeting the unmet need in CIT for a drug to decrease the incidence of thrombocytopenia and the need for platelet transfusions in cancer patients.

As an adjuvant therapy to chemotherapy or radiation, HemaMax has unique properties, including, for example: 1) In addition to platelet recovery following chemotherapy or radiation, HemaMax's MOA results in multilineage blood recovery, including recovery of neutrophils and red blood cells; 2) HemaMax has known anti-tumor responses that can be synergistic with the primary therapy, namely chemotherapy or radiation; and 3) no other cytokine can deliver these two unique properties when used as an adjuvant.

The instant disclosure provided methods and compositions for treating and/or preventing a subject from chemotherapy-induced cytopenias following exposure of the subject to chemotherapeutic agents, the method comprising: administering a dose of therapeutically effective amount of a pharmaceutical composition comprising substantially isolated IL-12 to the subject, whereby cytopenias, are diminished.

As used herein, IL-12 is a heterodimeric cytokine, comprising both p40 and p35 subunits, that is well-known for its role in immunity. In numerous reports spanning about two decades, IL-12 has been shown to have an essential role in the interaction between the innate and adaptive arms of immunity by regulating inflammatory responses, innate resistance to infection, and adaptive immunity. Endogenous IL-12 is required for resistance to many pathogens and to transplantable and chemically induced tumors. The hallmark effect of IL-12 in immunity is its ability to stimulate the production of interferon-gamma (IFN-gamma) from natural killer (NK) cells, macrophages and T cells. Further, several in vitro studies in the early-mid nineties reported that IL-12 is capable of stimulating hematopoiesis synergistically with other cytokines. The hematopoiesis-promoting activity of IL-12 appears to be due to a direct action on bone marrow stem cells as these studies used highly purified progenitors or even single cells. The role of IFN-gamma in the hematopoietic activity of IL-12 is not clear as several studies have linked both the promotion and suppression of hematopoiesis to IFN-gamma.

Interleukin-12 (IL-12) is shown to have a radioprotective function when used before or shortly after exposure to total body radiation (Neta et al. (1994) IL-12 protects bone marrow from and sensitizes intestinal tract to ionizing radiation. J Immunol 153: 4230-4237; Chen et al, (2007) IL-12 facilitates both the recovery of endogenous hematopoiesis and the engraftment of stem cells after ionizing radiation, Exp Hematol 35: 203-213; Basile et al. (2008) Multilineage hematopoietic recovery with concomitant antitumor effects using low dose Interleukin-12 in myelosuppressed tumor-bearing mice, J. Trans. Med. 6(26); Gluzman-Poltorak et al., (2014) Randomized comparison of single dose of recombinant human IL-12 versus placebo for restoration of hematopoiesis and improved survival in rhesus monkeys exposed to lethal radiation, J. Hematol. Oncol. 7(31); Gluzman-Poltorak et al., (2014) Recombinant interleukin-12, but not granulocyte-colony stimulating factor, improves survival in lethally irradiated nonhuman primates in the absence of supportive care: Evidence for the development of a frontline radiation medical countermeasure, Am. J. Hematol., 00(00); Gokhale et al., (2014) Single low-dose rHuIL-12 safely triggers multilineage hematopoietic and immune-mediated effects, Exp. Hematol. Oncol., 3(11); in addition, the entire disclosures of US20110206635 and U.S. Pat. No. 7,939,058 are herein incorporated by reference. In the studies, mice were rescued from the deleterious effects of lethal total body radiation. The radioprotective effect was reported to reside within an unknown cell population in the bone marrow, likely long-term repopulating hematopoietic stem cells. In another study, IL-12 was shown to provide early recovery peripheral blood cell counts following sublethal radiation of tumor-bearing mice (Basile et al. (2008) Multilineage hematopoietic recovery with concomitant antitumor effects using low dose Interleukin-12 in myelosuppressed tumor-bearing mice. J Transl Med 6: 26). In this latter study, it was shown that IL-12 was synergistic with radiation in reducing tumor volume. In particular, IL-12 did not to increase tumor volumes when administered either before or after radiation exposure.

Thus, IL-12 has potential in radioprotection of the bone marrow following total body radiation. However, early studies reported that although IL-12 had a radioprotective effect in the bone marrow, the gastrointestinal (GI) system was sensitized to radiation damage (Neta et al.). In a later report, the GI sensitization effect of IL-12 was found to be dependent on the dose of IL-12 administered (Chen et al.). There have been no reports of the radioprotective effects of IL-12 to other tissues or organs, other than bone marrow.

The present invention is based a surprising and unexpected discovery that certain murine recombinant IL-12 (e.g. m-HemaMax) and human recombinant IL-12 (e.g. HemaMax) have the ability to treat or prevent cytopenias, including CIT, in a subject in need thereof.

Leading hematopoietic supportive care therapies (EPO) have received black box warnings in response to their effect on tumor growth. The direct mechanism of action of HemaMax on hematopoietic stem cells can be contrasted with other well-known hematopoietic growth factors, such as EPO (branded as Procrit, Aranesp, and Epogen), and G-CSF (branded as Neulasta and Neupogen), as well as TPO mimetics (branded as Nplate and Promacta) and IL-11 (branded as Neumega). EPO-like molecules act at the level of erythroid precursor cells yielding increases in red blood cells. G-CSF-like molecules act at the level of neutrophil precursor cells yielding increases in neutrophils. TPO mimetics and IL-11 act at the level of megakaryoctes leading to increases in platelets. Target cell populations of these hematopoietic growth factors are all downstream of the hematopoietic stem cell, which is HemaMax's target cell.

There is no overlap between HemaMax's mechanism of action and that of the well-known hematopoeitic growth factors. HemaMax's mechanism of action involves activation of hematopoietic stem cells upstream of the activity of other hematopoietic factors. Consequently, HemaMax can replenish and regenerate the hematopoietic and immune systems following ablation, whereas these downstream acting factors cannot, as they target precursor cells to yield a single blood cell type. Via this early-acting (upstream) mechanism, HemaMax's activation of primitive hematopoietic stem cells can restore all major blood cell types. In pre-clinical studies, HemaMax has anti-tumor effects given its immunotherapy mechanism of action (increase in INF-γ and upregulation of T and NK cells).

In one aspect, the murine counterpart to HemaMax (rMuIL-12) has shown unexpected and surprising efficacy in preventing and/or treating cytopenias, including CIT.

In one aspect, the murine counterpart to HemaMax (rMuIL-12) promotes full lineage blood cell recovery including white and red blood cells and platelets in both normal and tumor-bearing mice exposed to sublethal or lethal Total Body Irradiation (TBI). The activity of HemaMax is initiated at the level of primitive cells (hematopoietic and non-hematopoietic stem cells) residing in the bone marrow compartment. Activation of these primitive cells leads to regeneration of the bone marrow compartment following myeloablation or myelosuppression caused by radiation or chemotherapy.

For the purpose of the current disclosure, the following definitions shall in their entireties be used to define technical terms and to define the scope of the composition of matter for which protection is sought in the claims.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, apes, and prenatal, pediatric, and adult humans.

As used herein, “preventing” or “protecting” means preventing in whole or in part, or ameliorating or controlling.

As used herein, the term “treating” refers to both therapeutic treatment and prophylactic or preventative measures, or administering an agent suspected of having therapeutic potential.

The term “a pharmaceutically effective amount” as used herein means an amount of active compound or pharmaceutical agent that elicits the biological or medicinal response in a tissue, system, animal or human that is being sought by a researcher, veterinarian, medical doctor or other clinician, which includes alleviation or palliation of the symptoms of the disease being treated.

As used herein, an “effective amount” in reference to the pharmaceutical compositions of the instant disclosure refers to the amount sufficient to have utility and provide desired therapeutic endpoint.

As used herein, radiation induced damage following total body irradiation (TBI) can affect organ, tissues, systems associated with the following: bone marrow, lymphatic system, immune system, mucosal tissue, mucosal immune system, gastrointestinal system, cardiovascular system, nervous system, reproductive organs, prostate, ovaries, lung, kidney, skin and brain.

As used herein, radiation exposure may be associated with radiation-induced acute, chronic, and systemic damage effects. In one aspect, the instant disclosure provides therapeutic compositions and methods of use thereof for treating radiation induced acute damage effects. Exemplary damage effects are not always limited to the normal tissue in the irradiation beam. Exemplary damage effect can extend beyond the treated area and can include, for example, esophagitis (difficulty swallowing); pneumonitis (cough, fever, lung fluid accumulation) in the lung; intestinal irradiation-induced inflammation (diarrhea, cramps, abdominal pain); nausea and vomiting; tiredness, fatigue, diarrhea, headache, tissue swelling, skin erythema, cough, and difficulty breathing. Exemplary damage effects can affect areas of the skin e.g. erythema, desquamation; oral mucosa, e.g. mucositis, nasopharynx; oropharynx; vocal cord; tonsil; skin, (squamous or carcinoma). In certain embodiments, exemplary effects can include telangiectasia, fibrosis, spinal cord myelitis, and cartilage fibrosis.

In certain embodiments, exemplary radiation induced damage effects can also include Blood-forming organ (Bone marrow) syndrome, characterized by damage to cells that divide at the most rapid pace (such as bone marrow, the spleen and lymphatic tissue). Exemplary symptoms include internal bleeding, fatigue, bacterial infections, and fever.

In certain embodiments, exemplary radiation induced damage effects can also include gastrointestinal tract syndrome, characterized by damage to cells that divide less rapidly (such as the linings of the stomach and intestines). Exemplary symptoms include nausea, vomiting, diarrhea, dehydration, electrolytic imbalance, loss of digestion ability, bleeding ulcers, and the symptoms of blood-forming organ syndrome.

In certain embodiments, exemplary radiation-induced damage effects can also include mucositis. In one embodiment, the radiation-induced mucositis is oral mucositis.

In certain embodiments, exemplary radiation induced effects can also include central nervous system syndrome, characterized by damage to cells that do not reproduce such as nerve cells. Exemplary symptoms include loss of coordination, confusion, coma, convulsions, shock, and the symptoms of the blood forming organ and gastrointestinal tract syndromes.

In certain embodiments, exemplary radiation induced damage effects can also include effects on the fetus due to prenatal radiation exposure. An embryo/fetus is especially sensitive to radiation, (embryo/fetus cells are rapidly dividing), particularly in the first 20 weeks of pregnancy.

In certain embodiments, exemplary radiation induced effects can also include damages due to ionizing irradiation-induced production of radical oxygen species (ROS) including superoxide, hydroxyl radical, nitric oxide and peroxynitrite from the interaction of ionizing irradiation with oxygen and water.

In one aspect, the instant disclosure provides therapeutic compositions and methods of use thereof for treating radiation induced chronic damage effects. Chronic irradiation effects are critically important in all patients, but particularly in those who receive total body irradiation (TBI). Total body irradiation is utilized in some cancer therapies particularly for patients who require a bone marrow transplant.

Exemplary radiation induced chronic damage effects can include, for example, features common to premature aging such as hair graying, skin thinning and dryness, formation of cataracts, early myocardial fibrosis, myocardial infarction, neurodegeneration, osteopenia/osteomalasia and neurocognitive defects.

In certain embodiments, exemplary radiation induced effects can also include fibrosis (the replacement of normal tissue with scar tissue, leading to restricted movement of the affected area); damage to the bowels, causing diarrhea and bleeding; memory loss; infertility and/or carcinogenesis/leukemogenesis.

In certain embodiments, the methods and compositions of the present disclosure are useful for improving hematopoiesis following stem cell transplantation. Exemplary myeloablative delivery modality/regimen can include, for example, conventional fractionation therapy, hyperfractionation, hypofractionation, and accelerated fractionation.

In one embodiment, the therapeutic modality/regimen is hyperfractionation therapy. In hyperfractionation, the goal is to deliver higher tumor doses while maintaining a level of long-term tissue damage that is clinically acceptable. The daily dose is unchanged or slightly increased while the dose per fraction is decreased, and the overall treatment time remains constant.

In one embodiment, the therapeutic modality/regimen is accelerated fractionation therapy. In the accelerated fractionation therapy, the dose per fraction is unchanged while the daily dose is increased, and the total time for the treatment is reduced.

In one embodiment, the therapeutic modality/regimen is Continuous hyperfractionated accelerated radiation therapy (CHART) therapy. In (CHART) therapy, an intense schedule of treatment in which multiple daily fractions are administered within an abbreviated period.

In one embodiment, the therapeutic modality/regimen is IMRT.

Chemotherapy Modalities

There are a number of strategies in the administration of chemotherapeutic drugs used today. Chemotherapy may be given with a curative intent or it may aim to prolong life or to palliate symptoms.

Combined modality chemotherapy is the use of drugs with other cancer treatments, such as radiation therapy or surgery. Most cancers are now treated in this way. Combination chemotherapy is a similar practice that involves treating a patient with a number of different drugs simultaneously. The drugs differ in their mechanism and side effects. The biggest advantage is minimizing the chances of resistance developing to any one agent.

In neoadjuvant chemotherapy (preoperative treatment) initial chemotherapy is designed to shrink the primary tumor, thereby rendering local therapy (surgery or radiotherapy) less destructive or more effective.

Adjuvant chemotherapy (postoperative treatment) can be used when there is little evidence of cancer present, but there is risk of recurrence. This can help reduce chances of relapse. It is also useful in killing any cancerous cells that have spread to other parts of the body. This is often effective as the newly growing tumours are fast-dividing, and therefore very susceptible. Palliative chemotherapy is given without curative intent, but simply to decrease tumor load and increase life expectancy. For these regimens, a better toxicity profile is generally expected.

As used herein, chemotherapy regimens require that the patient be capable of undergoing the treatment. Performance status is often used as a measure to determine whether a patient can receive chemotherapy, or whether dose reduction is required. Because only a fraction of the cells in a tumor die with each treatment (fractional kill), repeated doses must be administered to continue to reduce the size of the tumor. Current chemotherapy regimens apply drug treatment in cycles, with the frequency and duration of treatments limited by toxicity to the patient.

Types of Chemotherapeutic Agents

As used herein, the majority of chemotherapeutic drugs can be divided in to alkylating agents, antimetabolites, anthracyclines, plant alkaloids, topoisomerase inhibitors, and other antitumour agents. All of these drugs affect cell division or DNA synthesis and function in some way.

In certain embodiments, chemotherapeutic agents do not directly interfere with DNA. These include monoclonal antibodies and the tyrosine kinase inhibitors e.g. imatinib mesylate (Gleevec or Glivec), which directly targets a molecular abnormality in certain types of cancer (chronic myelogenous leukemia, gastrointestinal stromal tumors). These are examples of targeted therapies.

In other embodiments, certain drugs that modulate tumor cell behavior without directly attacking those cells may be used. Hormone treatments fall into this category.

Alkylating Agents

Alkylating Antineoplastic Agent

Alkylating agents are so named because of their ability to alkylate many nucleophilic functional groups under conditions present in cells. Cisplatin and carboplatin, as well as oxaliplatin, are alkylating agents. They impair cell function by forming covalent bonds with the amino, carboxyl, sulfhydryl, and phosphate groups in biologically important molecules.

Other agents are mechlorethamine, cyclophosphamide, chlorambucil, ifosfamide. They work by chemically modifying a cell's DNA.

Anti-Metabolites

Anti-metabolites masquerade as purines (azathioprine, mercaptopurine) or pyrimidines—which become the building-blocks of DNA. They prevent these substances from becoming incorporated in to DNA during the “S” phase (of the cell cycle), stopping normal development and division. They also affect RNA synthesis. Due to their efficiency, these drugs are the most widely used cytostatics.

Plant Alkaloids and Terpenoids

These alkaloids are derived from plants and block cell division by preventing microtubule function. Microtubules are vital for cell division, and, without them, cell division cannot occur. The main examples are vinca alkaloids and taxanes.

Vinca Alkaloids

Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea). The vinca alkaloids include: Vincristine; Vinblastine; Vinorelbine; and Vindesine.

Podophyllotoxin

Podophyllotoxin is a plant-derived compound that is said to help with digestion as well as used to produce two other cytostatic drugs, etoposide and teniposide. They prevent the cell from entering the GI phase (the start of DNA replication) and the replication of DNA (the S phase). The exact mechanism of its action is not yet known.

The substance has been primarily obtained from the American Mayapple (Podophyllum peltatum). Recently it has been discovered that a rare Himalayan Mayapple (Podophyllum hexandrum) contains it in a much greater quantity, but, as the plant is endangered, its supply is limited. Studies have been conducted to isolate the genes involved in the substance's production, so that it could be obtained recombinantly.

Taxanes

The prototype taxane is the natural product paclitaxel, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

Topoisomerase Inhibitors

Topoisomerases are essential enzymes that maintain the topology of DNA. Inhibition of type I or type II topoisomerases interferes with both transcription and replication of DNA by upsetting proper DNA supercoiling.

Some type I topoisomerase inhibitors include camptothecins: irinotecan and topotecan.

Examples of type II inhibitors include amsacrine, etoposide, etoposide phosphate, and teniposide. These are semisynthetic derivatives of epipodophyllotoxins, substances naturally occurring in the root of American Mayapple (Podophyllum peltatum).

Cytotoxic antibiotics: these include for example, actinomycin; anthracyclines; doxorubicin; daunorubicim; valrubicin; idarubicin; epirubicin, which also inhibit topoisomerase II.

Other cytotoxic antibiotics may include for example, bleomycin. Bleomycin acts in unique way through oxidation of a DNA-bleomycin-Fe(II) complex and forming free radicals, which induce damage and chromosomal aberrations. Other exemplars include Plicamycin and mitomycin.

A number of chemotherapeutic agents can enhance the effects of radiation therapy. In one aspect, the aspects and embodiments of the present disclosure can be utilized as a combined therapy with existing chemotherapeutic modalities. The combination (sequential or concurrent) therapy can be co-administration or co-formulation.

“Interleukin-12 (IL-12)” refers to IL-12 molecule that yields at least one of the hematopoietic properties disclosed herein, including native IL-12 molecules, variant 11-12 molecules and covalently modified IL-12 molecules, now known or to be developed in the future, produced in any manner known in the art now or to be developed in the future.

The IL-12 molecule may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, including, for example, at least about 95%, at least about 98% or at least about 99% of the peptide or dry mass of the preparation.

Generally, the amino acid sequences of the IL-12 molecule used in embodiments of the invention are derived from the specific mammal to be treated by the methods of the invention. Thus, for the sake of illustration, for humans, generally human IL-12, or recombinant human IL-12, would be administered to a human in the methods of the invention, and similarly, for felines, for example, the feline IL-12, or recombinant feline IL-12, would be administered to a feline in the methods of the invention.

Also included in the invention, however, are certain embodiments where the IL-12 molecule does not derive its amino acid sequence from the mammal that is the subject of the therapeutic methods of the invention. For the sake of illustration, human IL-12 or recombinant human IL-12 may be utilized in a feline mammal. Still other embodiments of the invention include IL-12 molecules where the native amino acid sequence of IL-12 is altered from the native sequence, but the IL-12 molecule functions to yield the hematopoietic properties of IL-12 that are disclosed herein. Alterations from the native, species-specific amino acid sequence of IL-12 include changes in the primary sequence of IL-12 and encompass deletions and additions to the primary amino acid sequence to yield variant IL-12 molecules. An example of a highly derivatized IL-12 molecule is the redesigned IL-12 molecule produced by Maxygen, Inc. (Leong S R, et al., Proc Natl Acad Sci USA. 2003 Feb. 4; 100 (3): 1163-8), where the variant IL-12 molecule is produced by a DNA shuffling method. Also included are modified IL-12 molecules are also included in the methods of invention, such as covalent modifications to the IL-12 molecule that increase its shelf life, half-life, potency, solubility, delivery, etc., additions of polyethylene glycol groups, polypropylene glycol, etc., in the manner set forth in U.S. Pat. No. 4,640,835; 4,496,689; 4,301,144; 4,670,417; 4,791,192 or 4,179,337. One type of covalent modification of the IL-12 molecule is introduced into the molecule by reacting targeted amino acid residues of the IL-12 polypeptide with an organic derivatizing agent that is capable of reacting with selected side chains or the N- or C-terminal residues of the IL-12 polypeptide. Both native sequence IL-12 and amino acid sequence variants of IL-12 may be covalently modified. Also as referred to herein, the IL-12 molecule can be produced by various methods known in the art, including recombinant methods. Other IL-12 variants included in the present disclosure are those where the canonical sequence is post-translationally-modified, for example, glycosylated. In certain embodiments, the IL-12 is expressed in a mammalian expression system or cell line. In one embodiment, the IL-12 is produced by expression in Chinese Hamster Ovary (CHO) cells.

Since it is often difficult to predict in advance the characteristics of a variant IL-12 polypeptide, it will be appreciated that some screening of the recovered variant will be needed to select the optimal variant. A preferred method of assessing a change in the hematological stimulating or enhancing properties of variant IL-12 molecules is via the lethal irradiation rescue protocol disclosed below. Other potential modifications of protein or polypeptide properties such as redox or thermal stability, hydrophobicity, susceptibility to proteolytic degradation, or the tendency to aggregate with carriers or into multimers are assayed by methods well known in the art.

For general descriptions relating IL-12, see U.S. Pat. Nos. 5,573,764, 5,648,072, 5,648,467, 5,744,132, 5,756,085, 5,853,714 and 6,683,046. Interleukin-12 (IL-12) is a heterodimeric cytokine generally described as a proinflammatory cytokine that regulates the activity of cells involved in the immune response (Fitz K M, et al., 1989, J. Exp. Med. 170:827-45). Generally IL-12 stimulates the production of interferon-γ (INF-γ) from natural killer (NK) cells and T cells (Lertmemongkolchai G, Cai et al., 2001, Journal of Immunology. 166:1097-105; Cui J, Shin T, et al., 1997, Science. 278:1623-6; Ohteki T, Fukao T, et alk., 1999, J. Exp. Med. 189:1981-6; Airoldi I, Gri G, et al., 2000, Journal of Immunology. 165:6880-8), favors the differentiation of T helper 1 (TH1) cells (Hsieh C S, et al., 1993, Science. 260:547-9; Manetti R, et al., 1993, J. Exp. Med. 177:1199-1204), and forms a link between innate resistance and adaptive immunity. IL-12 has also been shown to inhibit cancer growth via its immunomodulatory and anti-angiogenesis effects (Brunda M J, et al., 1993, J. Exp. Med. 178:1223-1230; Noguchi Y, et al., 1996, Proc. Natl. Acad. Sci. U.S.A. 93:11798-11801; Giordano P N, et al., 2001, J. Exp. Med. 194:1195-1206; Colombo M P, et al, 2002, Cytokine Growth factor rev. 13:155-168; Yao L, et al., 2000, Blood 96:1900-1905). IL-12 is produced mainly by dendritic cells (DC) and phagocytes (macrophages and neutrophils) once they are activated by encountering pathogenic bacteria, fungi or intracellular parasites (Reis C, et al., 1997, J. Exp. Med. 186:1819-1829; Gazzinelli R T, et al., 1994, J. Immunol. 153:2533-2543; Dalod M, et al., 2002, J. Exp. Med. 195:517-528). The IL-12 receptor (IL-12 R) is expressed mainly by activated T cells and NK cells (Presky D H, et al., 1996, Proc. Natl. Acad. Sci. U.S.A. 93:14002-14007; Wu C Y, et al., 1996, Eur J. Immunol. 26:345-50).

Generally the production of IL-12 stimulates the production of INF-γ, which, in turn, enhances the production of IL-12, thus forming a positive feedback loop. In in vitro systems, it has been reported that IL-12 can synergize with other cytokines (IL-3 and SCF for example) to stimulate the proliferation and differentiation of early hematopoietic progenitors (Jacobsen S E, et al., 1993, J. Exp Med 2: 413-8; Ploemacher R E, et al., 1993, Leukemia 7: 1381-8; Hirao A, et al., 1995, Stem Cells 13: 47-53).

In vivo administration of IL-12 was observed to decrease peripheral blood cell counts and bone marrow hematopoiesis (Robertson M J, et al., 1999, Clinical Cancer Research 5: 9-16; Lenzi R, et al., 2002, Clinical Cancer Research 8:3686-95; Ryffel B. 1997, Clin Immunol Immunopathol. 83:18-20; Car B D, et al., 1999, The Toxicol Pathol. 27:58-63). Using INF-γ receptor knockout mice, Eng et al and Car et al demonstrated that high dose IL-12 did not induce the commonly seen toxicity effect, i.e., there was no inhibition of hematopoiesis (Eng V M, et al., 1995, J. Exp Med. 181:1893-8; Car B D, et al., 1995, American Journal of Pathology 147:1693-707). This observation suggests that the general phenomenon of IL-12 facilitated enhancement of differentiated hematopoietic cells, as reported previously, may be balanced in vivo by the production of INF-γ, which acts in a dominant myelo-suppressive fashion.

Current evidence suggests that an exemplary IL-12 preparation, a recombinant human IL-12 (e.g., HemaMax), triggers responses at, at least, 4 levels in the body (see FIG. 14). At the Level 1 response, HemaMax promotes proliferation and activation of extant, radiosensitive immune cells, namely NK cells, macrophages, and dendritic cells. HemaMax-induced plasma elevations of IL-15 and IL-18 also facilitate maturation of NK cells, leading to the release of IFN-γ, which in turn, positively affects the production of endogenous IL-12 from macrophages and dendritic cells, and perhaps NK cells. These events enhance the innate immune competency early on following HemaMax administration. At the Level 2 response, HemaMax promotes proliferation and differentiation of the surviving hematopoietic stem cells, osteoblasts, and megakaryocytes into a specific cellular configuration that ensues optimal hematopoiesis. HemaMax-induced secretion of EPO from CD34+, IL-12Rβ2-positive bone marrow cells may also suppress local over-production of IFN-γ in the bone marrow and, thus, provide a milieu that promotes expansion of hematopoietic cells. Hematopoietic regeneration in the bone marrow enhances both innate and adaptive immune competency. At the Level 3 response, HemaMax preserves GI stem cells, leading to a reduction in pathogen leakage, an increase in food consumption, and a decrease in diarrhea. At the Level 4 response, HemaMax likely directly increases renal release of EPO, a cytoprotective factor, which enhances cellular viability in a diverse set of organs/tissues. Continued production of endogenous IL-12 primarily from dendritic cells activated by pathogens and/or EPO serves as a positive feedback loop and plays a key role in sustaining the initial response to exogenous HemaMax, perhaps for weeks after radiation.

### Methods of Administration of IL-12

The instant disclosure provides methods of treatment by administration to a subject of one or more effective dose(s) of IL-12 for a duration to achieve the desired therapeutic effect. The subject is preferably a mammal, including, but not limited to, animals such as cows, pigs, horses, chickens, cats, dogs, etc., and is most preferably human.

Various delivery systems are known and can be used to administer IL-12 in accordance with the methods of the invention, e.g., encapsulation in liposomes, microparticles, microcapsules, recombinant cells capable of expressing IL-12, receptor-mediated endocytosis (see, e.g., Wu and Wu, 1987, J. Biol. Chem. 262:4429-4432), construction of nucleic acid comprising a gene for IL-12 as part of a retroviral or other vector, etc. Methods of introduction include but are not limited to intradermal, intramuscular, intraperitoneal, intravenous, subcutaneous, intranasal, epidural, and oral routes.

IL-12 can be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce pharmaceutical compositions comprising IL-12 into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. It may be desirable to administer the pharmaceutical compositions comprising IL-12 locally to the area in need of treatment; this may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

Other modes of IL-12 administration involve delivery in a vesicle, in particular a liposome (see Langer, Science 249:1527-1533 (1990): Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

Still other modes of administration of IL-12 involve delivery in a controlled release system. In certain embodiments, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). Additionally polymeric materials can be used (see Medical Applications of Controlled Release, Langer and Wise (eds.), CRC Pres, Boca Raton, Fla. (1974); Controlled Drug Bioavailability, Drug Product Design and Performance, Smolen and Ball (eds.), Wiley. N.Y. (1984); Ranger and Peppas, J. Macromol. Sci. Rev. Macromol. Chem. 23:61 (1983; see also Levy et al., Science 228:190 (1985); During et al., Ann. Neurol. 25:351 (1989); Howard et al., J. Neurosurg. 71:105 (1989)), or a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In one aspect, the one or more effective dose(s) of IL-12 are given before chemotherapy exposure. In other aspects, the one or more effective dose(s) of IL-12 are given before, during and/or after chemotherapy exposure. In another aspect, the one or more effective dose(s) of IL-12 are given after chemotherapy exposure.

In certain aspects, the one or more effective dose(s) of IL-12 is given at greater than about 24, about 48, about 72, about 96 or about 120 hours after chemotherapy exposure.

In one aspect, the one or more effective doses of IL-12 are administered topically, subcutaneously, intradermally, intravenously, intraperitoneally, intramuscularly, epidurally, parenterally, intranasally, and/or intracranially.

Forms and Dosages of IL-12

Suitable dosage forms of IL-12 for use in embodiments of the present invention encompass physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of IL-12 polypeptides include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Suitable examples of sustained-release preparations include semipermeable matrices of solid hydrophobic polymers containing the polypeptide, which matrices are in the form of shaped articles, e.g. films, or microcapsules. Examples of sustained-release matrices include polyesters, hydrogels (for example, poly(2-hydroxyethyl-methacrylate) as described by Langer et al., supra and Langer, supra, or poly(vinylalcohol), polylactides (U.S. Pat. No. 3,773,919), copolymers of L-glutamic acid and gamma ethyl-L-glutamate (Sidman et al, supra), non-degradable ethylene-vinyl acetate (Langer et al., supra), degradable lactic acid-glycolic acid copolymers such as the Lupron Depot™ (injectable microspheres composed of lactic acid-glycolicacid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated IL-12 polypeptides remain in the body for a long time, they may denature or aggregate as a result of exposure to moisture at 37 degree C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release IL-12 containing compositions also include liposomally entrapped polypeptides. Liposomes containing a IL-12 polypeptide are prepared by methods known in the art, such as described in Eppstein et al., Proc. Natl. Acad. Sci. USA 82:3688-3692 (1985); Hwang et al., Proc. Natl. Acad. Sci. USA 77:4030 (1980); and U.S. Pat. Nos. 4,485,045 and 4,544,545. Ordinarily, the liposomes are the small (about 200-800 Angstroms) unilamelar type in which the lipid content is greater than about 30 mol. % cholesterol, the selected proportion being adjusted for the optimal Wnt polypeptide therapy. Liposomes with enhanced circulation time are disclosed in U.S. Pat. No. 5,013,556.

For the treatment of disease, the appropriate dosage of a IL-12 polypeptide will depend on the type of disease to be treated, as defined above, the severity and course of the disease, previous therapy, the patient's clinical history and response to the IL-12 therapeutic methods disclosed herein, and the discretion of the attending physician. In accordance with the invention, IL-12 is suitably administered to the patient at one time or over a series of treatments.

Depending on the type and severity of the disease, about 10 ng/kg to 2000 ng/kg of IL-12 is an initial candidate dosage for administration to the patient, whether, for example, by one or more separate administrations, or by continuous infusion. Humans can safely tolerate a repeated dosages of about 500 ng/kg, but single dosages of up to about 200 ng/kg should not produce toxic side effects. For example, the dose may be the same as that for other cytokines such as G-CSF, GM-CSF and EPO. For repeated administrations over several days or longer, depending on the condition, the treatment is sustained until a desired suppression of disease symptoms occurs. However, other dosage regimens may be useful. The progress of this therapy is easily monitored by conventional techniques and assays.

IL-12 may be administered along with other cytokines, either by direct co-administration or sequential administration. When one or more cytokines are co-administered with IL-12, lesser doses of IL-12 may be employed. Suitable doses of other cytokines, i.e. other than IL-12, are from about 1 ug/kg to about 15 mg/kg of cytokine. For example, the dose may be the same as that for other cytokines such as G-CSF, GM-CSF and EPO. The other cytokine(s) may be administered prior to, simultaneously with, or following administration of IL-12. The cytokine(s) and IL-12 may be combined to form a pharmaceutically composition for simultaneous administration to the mammal. In certain embodiments, the amounts of IL-12 and cytokine are such that a synergistic repopulation of blood cells (or synergistic increase in proliferation and/or differentiation of hematopoietic cells) occurs in the mammal upon administration of IL-12 and other cytokine thereto. In other words, the coordinated action of the two or more agents (i.e. the IL-12 and one or more cytokine(s)) with respect to repopulation of blood cells (or proliferation/differentiation of hematopoietic cells) is greater than the sum of the individual effects of these molecules.

Therapeutic formulations of IL-12 are prepared for storage by mixing IL-12 having the desired degree of purity with optional physiologically acceptable carriers, excipients, or stabilizers (Remington's Pharmaceutical Sciences, 16th edition, Osol, A., Ed., (1980)), in the form of lyophilized cake or aqueous solutions. Acceptable carriers, excipients, or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or non-ionic surfactants such as Tween®, Pluronics™ or polyethylene glycol (PEG).

The term “buffer” as used herein denotes a pharmaceutically acceptable excipient, which stabilizes the pH of a pharmaceutical preparation. Suitable buffers are well known in the art and can be found in the literature. Pharmaceutically acceptable buffers include but are not limited to histidine-buffers, citrate-buffers, succinate-buffers, acetate-buffers, phosphate-buffers, arginine-buffers or mixtures thereof. The abovementioned buffers are generally used in an amount of about 1 mM to about 100 mM, of about 5 mM to about 50 mM and of about 10-20 mM. The pH of the buffered solution can be at least 4.0, at least 4.5, at least 5.0, at least 5.5 or at least 6.0. The pH of the buffered solution can be less than 7.5, less than 7.0, or less than 6.5. The pH of the buffered solution can be about 4.0 to about 7.5, about 5.5 to about 7.5, about 5.0 to about 6.5, and about 5.5 to about 6.5 with an acid or a base known in the art, e.g. hydrochloric acid, acetic acid, phosphoric acid, sulfuric acid and citric acid, sodium hydroxide and potassium hydroxide. As used herein when describing pH, “about” means plus or minus 0.2 pH units.

As used herein, the term “surfactant” can include a pharmaceutically acceptable excipient which is used to protect protein formulations against mechanical stresses like agitation and shearing. Examples of pharmaceutically acceptable surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Suitable surfactants include polyoxyethylenesorbitan-fatty acid esters such as polysorbate 20, (sold under the trademark Tween 20®) and polysorbate 80 (sold under the trademark Tween 80®). Suitable polyethylene-polypropylene copolymers are those sold under the names Pluronic® F68 or Poloxamer 188®. Suitable Polyoxyethylene alkyl ethers are those sold under the trademark Brij®. Suitable alkylphenolpolyoxyethylene esthers are sold under the tradename Triton-X. When polysorbate 20 (Tween 20®) and polysorbate 80 (Tween 80®) are used they are generally used in a concentration range of about 0.001 to about 1%, of about 0.005 to about 0.2% and of about 0.01% to about 0.1% w/v (weight/volume).

As used herein, the term “stabilizer” can include a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al., Crit. Rev. Ther. Drug Carrier Syst., 70(4):307-77 (1993); Wang, Int. J. Pharm., 7S5(2): 129-88 (1999); Wang, Int. J. Pharm., 203(1-2): 1-60 (2000); and Chi et al, Pharm. Res., 20(9): 1325-36 (2003). Stabilizers include but are not limited to sugars, amino acids, polyols, cyclodextrines, e.g. hydroxypropyl-beta-cyclodextrine, sulfobutylethyl-beta-cyclodextrin, beta-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, an amount of about 10 to about 300 mM, or in an amount of about 100 mM to about 300 mM. In some embodiments, exemplary IL-12 can be dissolved in an appropriate pharmaceutical formulation wherein it is stable.

IL-12 also may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles, and nanocapsules), or in macroemulsions. Such techniques are disclosed in Remington's Pharmaceutical Sciences, supra.

IL-12 to be used for in vivo administration must be sterile. This is readily accomplished by filtration through sterile filtration membranes, prior to or following lyophilization and reconstitution. IL-12 is stored in lyophilized form or in solution. Therapeutic IL-12 compositions generally are placed into a container having a sterile access port, for example, an intravenous solution bag or vial having a stopper pierceable by a hypodermic injection needle.

When applied topically, IL-12 is suitably combined with other ingredients, such as carriers and/or adjuvants. There are no limitations on the nature of such other ingredients, except that they must be physiologically acceptable and efficacious for their intended administration, and cannot degrade the activity of the active ingredients of the composition. Examples of suitable vehicles include ointments, creams, gels, or suspensions, with or without purified collagen. The compositions also may be impregnated into transdermal patches, plasters, and bandages, preferably in liquid or semi-liquid form.

For obtaining a gel formulation, IL-12 formulated in a liquid composition may be mixed with an effective amount of a water-soluble polysaccharide or synthetic polymer such as PEG to form a gel of the proper viscosity to be applied topically. The polysaccharide that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; starch and fractionated starch; agar; alginic acid and alginates; gum arabic; pullullan; agarose; carrageenan; dextrans; dextrins; fructans; inulin; mannans; xylans; arabinans; chitosans; glycogens; glucans; and synthetic biopolymers; as well as gums such as xanthan gum; guar gum; locust bean gum; gum arable; tragacanth gum; and karaya gum; and derivatives and mixtures thereof. The preferred gelling agent herein is one that is inert to biological systems, nontoxic, simple to prepare, and not too runny or viscous, and will not destabilize the IL-12 molecule held within it.

Preferably the polysaccharide is an etherified cellulose derivative, more preferably one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. Most preferred herein is methylcellulose.

The polyethylene glycol useful for gelling is typically a mixture of low and high molecular weight PEGs to obtain the proper viscosity. For example, a mixture of a PEG of molecular weight 400-600 with one of molecular weight 1500 would be effective for this purpose when mixed in the proper ratio to obtain a paste.

The term “water soluble” as applied to the polysaccharides and PEGs is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

If methylcellulose is employed in the gel, preferably it comprises about 2-5%, more preferably about 3%, of the gel and IL-12 is present in an amount of about 300-1000 mg per ml of gel.

An effective amount of IL-12 to be employed therapeutically will depend, for example, upon the therapeutic objectives, the route of administration, and the condition of the patient. Accordingly, it is necessary for the therapist to titer the dosage and modify the route of administration as required to obtain the optimal therapeutic effect. Typically, the clinician will administer IL-12 until a dosage is reached that achieves the desired effect. A typical dosage for systemic treatment might range from about 10 ng/kg to up to 2000 ng/kg or more, depending on the factors mentioned above. In some embodiments, the dose ranges can be from about 1, 2, 3, 4, 5, 6, 7, 8, 9, 10, 11, 12, 13, 14, 15, 16, 17, 18, 19 to about 20; to about 30; to about 50; to about 100, to about 200, to about 300 or to about 500 ng/kg. In one aspect, the dose is less than 500 ng/kg, In another aspect, the dose is less than 300 ng/kg. In another aspect, the dose is less than about 200 ng/kg. In another aspect, the dose is less than about 100 ng/kg. In another aspect, the dose is less than about 50 ng/kg. In other aspects, the dose can range from about 10 to 300 ng/kg, 20 to 40 ng/kg, 25 to 35 ng/kg, 50 to 100 ng/kg.

In one aspect, exemplary therapeutic compositions described herein can be administered in fractionated doses. In one embodiment, the therapeutically effective dose is given before each fraction. In one embodiment, the therapeutically effective dose is given at about the same time as the administration of each chemotherapeutic dose or dose fraction. In one embodiment, the therapeutically effective dose is given before each fraction, ranging from 5, 10, 15, 20, 25, 30, 35, 40, 50, or 60 minutes before each fraction; or 2, 3, 4, 5, 6, 7, 8, 9, 10, 11, 12 hours after each fraction; or 1, 2, 3, 4, 5, 6, 7 days before each fraction. In one embodiment, the therapeutically effective dose is given after each fraction, ranging from 5, 10, 15, 20, 25, 30, 35, 40, 50, or 60 minutes after each fraction; or 2, 3, 4, 5, 6, 7, 8, 9, 10, 11, 12 hours after each fraction; or 1, 2, 3, 4, 5, 6, 7 days after each fraction; or once, twice, three times, 4 times, 5 times, 6 time, 7 times weekly, biweekly, or bimonthly, during or after the chemotherapeutic and/or combination chemotherapeutic/radiation treatment. In another embodiment, one or more exemplary doses of IL-12 is administered (1 to 100 ng/kg) at about 5, 10, 15, 20, 30, 40, 50, 60 min, 1, 2, 3, 4, 5, 6, 7, 8, 9, 10, 11, 12, 13, 14, 15, 16, 17, 18, 19, 20, 21, 22, 23, 24 hours, 1 day, 2 days, 3 days, 4 days, 5 days, 6 days, 7 days both before and after each chemotherapeutic dose.

As an alternative general proposition, the IL-12 receptor is formulated and delivered to the target site or tissue at a dosage capable of establishing in the tissue an IL-12 level greater than about 0.1 ng/cc up to a maximum dose that is efficacious but not unduly toxic. This intra-tissue concentration should be maintained if possible by the administration regime, including by continuous infusion, sustained release, topical application, or injection at empirically determined frequencies. The progress of this therapy is easily monitored by conventional assays.

“Near the time of administration of the treatment” refers to the administration of IL-12 at any reasonable time period either before and/or after the administration of the treatment, such as about one month, about three weeks, about two weeks, about one week, several days, about 120 hours, about 96 hours, about 72 hours, about 48 hours, about 24 hours, about 20 hours, several hours, about one hour or minutes. Near the time of administration of the treatment may also refer to either the simultaneous or near simultaneous administration of the treatment and IL-12, i.e., within minutes to one day.

“Chemotherapy” refers to any therapy that includes natural or synthetic agents now known or to be developed in the medical arts. Examples of chemotherapy include the numerous cancer drugs that are currently available. However, chemotherapy also includes any drug, natural or synthetic, that is intended to treat a disease state. In certain embodiments of the invention, chemotherapy may include the administration of several state of the art drugs intended to treat the disease state. Examples include combined chemotherapy with docetaxel, cisplatin, and 5-fluorouracil for patients with locally advanced squamous cell carcinoma of the head (Tsukuda, M. et al., Int J Clin Oncol. 2004 June; 9 (3): 161-6), and fludarabine and bendamustine in refractory and relapsed indolent lymphoma (Konigsmann M, et al., Leuk Lymphoma. 2004; 45 (9): 1821-1827).

As used herein, exemplary sources of therapeutic or accidental ionizing radiation can include, for example, alpha, beta, gamma, x-ray, and neutron sources.

“Radiation therapy” refers to any therapy where any form of radiation is used to treat the disease state. The instruments that produce the radiation for the radiation therapy are either those instruments currently available or to be available in the future.

“High dose treatment modalities” refer to treatments that are high sub-lethal or near lethal. High dose treatment modalities are intended to have an increased ability to achieve therapeutic endpoint, but generally possess increased associated toxicities. Further, generally high dose treatment modalities exhibit increased hematopoietic damage, as compared with conventional treatment modalities. The protocols for high dose treatment modalities are those currently used or to be used in the future.

As used herein, radiation therapy “treatment modality” can include both ionizing and non-ionizing radiation sources. Exemplary ionizing radiation treatment modality can include, for example, external beam radiotherapy; Intensity modulated radiation therapy (IMRT); Image Guided Radiotherapy (IGRT); X Irradiation (e.g. photon beam therapy); electron beam (e.g. beta irradiation); proton irradiation; high linear energy transfer (LET) particles; stereotactic radiosurgery; gamma knife; linear accelerator mediated frameless stereotactic radiosurgery; robot arm controlled x irradiation delivery system; radioisotope radiotherapy for organ specific or cancer cell specific uptake; radioisotope bound to monoclonal antibody for tumor targeted radiotherapy (or radioimmunotherapy, RIT); brachytherapy (interstitial or intracavity) high dose rate radiation source implantation; permanent radioactive seed implantation for organ specific dose delivery.

“A dose dense treatment regimen” is generally a treatment regimen whereby the treatment is repeated sequentially in an accelerated manner to achieve the desired treatment outcome, as compared with conventional treatment regimens. The methods of the invention facilitate the use of dose dense treatment regimens by reducing or ameliorating the associated hematopoietic toxicities of the treatment, thereby permitting dose dense treatment regimens to be utilized and increasing the rate of success in treating a particular disease state. (see generally, Hudis C A, Schmits N, Semin Oncol. 2004 June; 31 (3 Suppl 8): 19-26; Keith B et al., J Clin Oncol. 2004 Feb. 15; 22 (4): 749; author reply 751-3; Maurel J et al, Cancer. 2004 Apr. 1; 100 (7): 1498-506; Atkins C D, J Clin Oncol. 2004 Feb. 15; 22 (4): 749-50.)

“Chemoprotection or radioprotection” refers to protection from, or an apparent decrease in, the associated hematopoietic toxicity of a treatment intended to target the disease state.

As used herein, “Acute Radiation Syndrome” (ARS) (also known as radiation toxicity or radiation sickness), is characterized by an acute illness caused by receiving lethal or sublethal irradiation of the entire body (or most of the body) by a high dose of penetrating radiation in a very short period of time (e.g. a matter of minutes). Examples of people who suffered from ARS are the survivors of the Hiroshima and Nagasaki atomic bombs, the firefighters that first responded after the Chernobyl Nuclear Power Plant event in 1986, and some unintentional exposures to sterilization irradiators. In certain embodiments, the radiation dose associated with acute radiation syndrome is usually large (i.e., greater than 0.7 Gray (Gy) or 70 rads). In certain embodiments, mild symptoms may be observed with doses as low as 0.3 Gy or 30 rads.

As used herein, “acute damage effects” and “damage effects” can include radiation induced damage due to acute lethal and near lethal radiation dose.

“Solid tumors” generally refers to the presence of cancer of body tissues other than blood, bone marrow, or the lymphatic system.

“Hematopoietic disorders (cancers)” generally refers to the presence of cancerous cells originated from hematopoietic system.

“Ameliorate the deficiency” refers to a reduction in the hematopoietic deficiency, i.e., an improvement in the deficiency, or a restoration, partially or complete, of the normal state as defined by current medical practice. Thus, amelioration of the hematopoietic deficiency refers to an increase in, a stimulation, an enhancement or promotion of, hematopoiesis generally or specifically. Amelioration of the hematopoietic deficiency can be observed to be general, i.e., to increase two or more hematopoietic cell types or lineages, or specific, i.e., to increase one hematopoietic cell type or lineages.

“Bone marrow cells” generally refers to cells that reside in and/or home to the bone marrow compartment of a mammal. Included in the term “bone marrow cells” is not only cells of hematopoietic origin, including but not limited to hematopoietic repopulating cells, hematopoietic stem cell and/or progenitor cells, but any cells that may be derived from bone marrow, such as endothelial cells, mesenchymal cells, bone cells, neural cells, supporting cells (stromal cells), including but not limited to the associated stem and/or progenitor cells for these and other cell types and lineages.

“Hematopoietic cell type” generally refers to differentiated hematopoietic cells of various types, but can also include the hematopoietic progenitor cells from which the particular hematopoietic cell types originate from, such as various blast cells referring to all the cell types related to blood cell production, including stem cells, progenitor cells, and various lineage cells, such as myeloid cells, lymphoid cell, etc.

“Hematopoietic cell lineage” generally refers to a particular lineage of differentiated hematopoietic cells, such as myeloid or lymphoid, but could also refer to more differentiated lineages such as dendritic, erythroid, etc.

“IL-12 facilitated proliferation” of cells refers to an increase, a stimulation, or an enhancement of hematopoiesis that at least partially attributed to an expansion, or increase, in cells that generally reside or home to the bone marrow of a mammal, such as hematopoietic progenitor and/or stem cells, but includes other cells that comprise the microenvironment of the bone marrow niche.

“Stimulation or enhancement of hematopoiesis” generally refers to an increase in one or more hematopoietic cell types or lineages, and especially relates to a stimulation or enhancement of one or more hematopoietic cell types or lineages in cases where a mammal has a deficiency in one or more hematopoietic cell types or lineages.

“Hematopoietic long-term repopulating cells” are generally the most primitive blood cells in the bone marrow; they are the blood stem cells that are responsible for providing life-long production of the various blood cell types and lineages.

“Hematopoietic stem cells” are generally the blood stem cells; there are two types: “long-term repopulating” as defined above, and “short-term repopulating” which can produce “progenitor cells” for a short period (weeks, months or even sometimes years depending on the mammal).

“Hematopoietic progenitor cells” are generally the first cells to differentiate from (i.e., mature from) blood stem cells; they then differentiate (mature) into the various blood cell types and lineages.

“Hematopoietic support cells” are the non-blood cells of the bone marrow; these cells provide “support” for blood cell production. These cells are also referred to as bone marrow stromal cells.

“Bone marrow preservation” means the process whereby bone marrow that has been damaged by radiation, chemotherapy, disease or toxins is maintained at its normal, or near normal, state; “bone marrow recovery” means the process whereby bone marrow that has been damaged by radiation, chemotherapy, disease or toxins is restored to its normal, near normal state, or where any measurable improvement in bone marrow function are obtained; bone marrow function is the process whereby appropriate levels of the various blood cell types or lineages are produced from the hematopoietic (blood) stem cells.

“Bone marrow failure” is the pathologic process where bone marrow that has been damaged by radiation, chemotherapy, disease or toxins is not able to be restored to normal and, therefore, fails to produce sufficient blood cells to maintain proper hematopoiesis in the mammal.

### EXAMPLES

The invention is now described with reference to the following Examples. These Examples are provided for the purpose of illustration only, and the invention is not limited to these Examples, but rather encompasses all variations that are evident as a result of the teaching provided herein.

Prior to the experiments described herein, there was no published protocol that allows for compositions and methods comprising IL-12, including recombinant human interleukin-12 (IL-12) preparation for efficacious treatment of a broad range of cytopenias, including CIT, along with providing significant anti-tumor effects in cancer patients receiving chemotherapy

Aspects and embodiments of the instant disclosure stem from the unexpected discovery that certain IL-12 formulations and dosing regimens that have a surprising and unexpected utility and efficacy when administered to a subject in need of chemotherapy treatment. In particular, the optimal doses of IL-12 in the invention lead to dual effects that involve both reduction of cytopenias, including CIT, and reduction in the tumor burden and/or MRD.

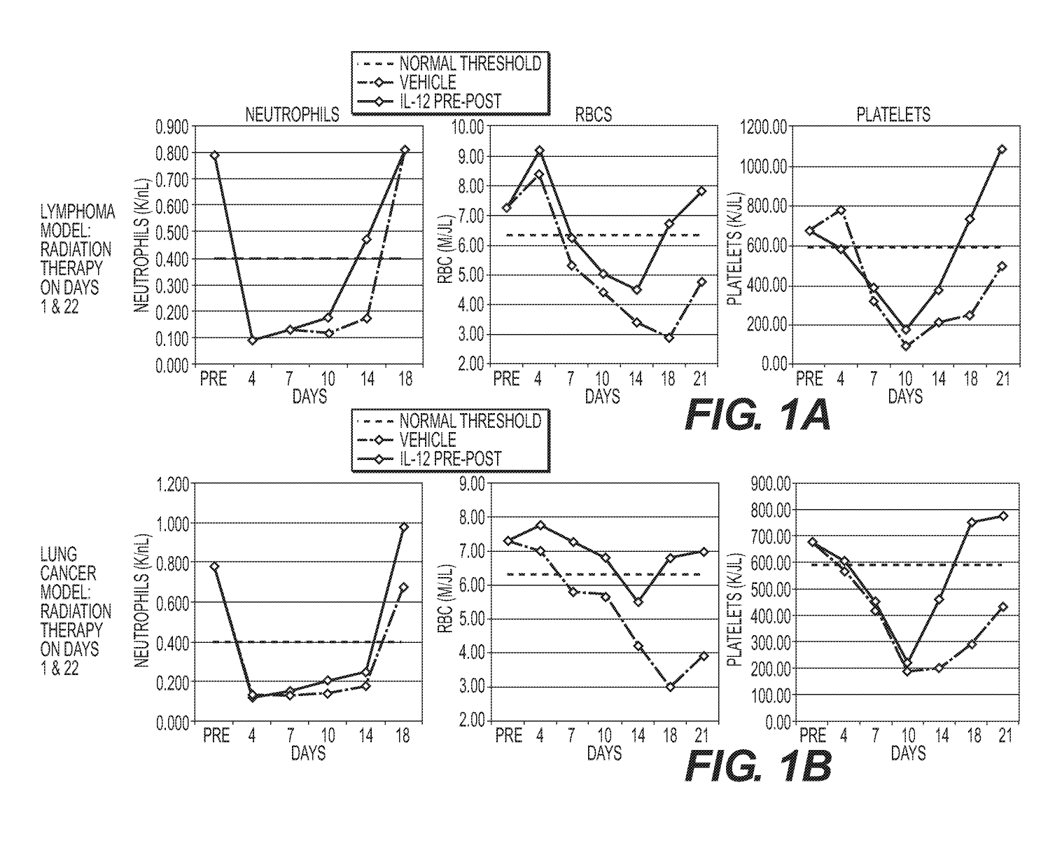

By way of example, methods and compositions for administering therapeutically effective IL-12 formulation for HIT therapy were developed. Supporting Data Related to the Efficacy of HemaMax™ in CIT are presented in the following, including FIGS. 1-13 and the examples of: studies in myelosuppressed, tumor-bearing mice; radiomitigation study in myeloablated, lethally irradiated nonhuman primates (NHP); the role of the IL-12 receptor in megakaryopoiesis; direct and indirect antitumor effects of IL-12; anti-thrombocytopenic effect of rMuIL-12 in murine models of CIT.

### Example 1

Studies in myelosuppressed, tumor-bearing mice demonstrated that Murine IL-12 facilitates early hematopoietic recovery with concomitant anti-tumor responses in myelosuppressed mice.

FIG. 1 A-B showed the Blood Recovery Profiles for Vehicle and rMuIL-12 Administered Before and After Myelosuppresive Radiation (625 rad). Platelet recovery profiles are shown for the EL4 lymphoma tumor model (a) and the Lewis lung cancer model (b). During days 14-21 in both tumor models, rMuIL-12-treated mice showed statistically significant improvements in platelet counts as compared to the vehicle control.

### Example 2

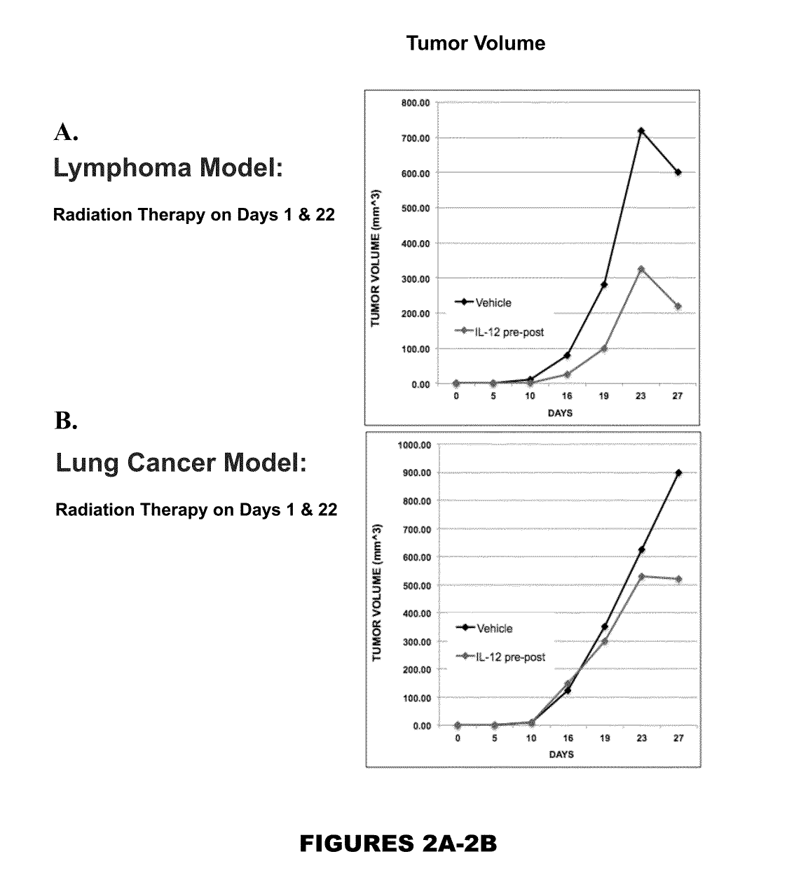

FIG. 2 A-B describe Relative Changes in Tumor Volumes for Vehicle and rMuIL-12 Treatment Groups Following Radiation (625 rad). Changes in tumor volumes over the course of the experiment are shown for the EL4 lymphoma tumor model (a) and the Lewis Lung cancer tumor model (b). Mice in both tumor models were given 625 rad on day 1. Mice in both tumor models were given a second dose of radiation on day 22 following the initial radiation dose. In the EL4 lymphoma model, all rMuIL-12 treatment groups, namely pre, post and pre-post radiation dosing groups, significantly reduced tumor growth (% T/C<50%) as compared with the control at the endpoint of tumor volume evaluation. In the Lewis lung cancer model, rMuIL-12 post-treatment significantly reduced tumor growth (% T/C<50%) at the endpoint of tumor growth evaluation.

### Example 3

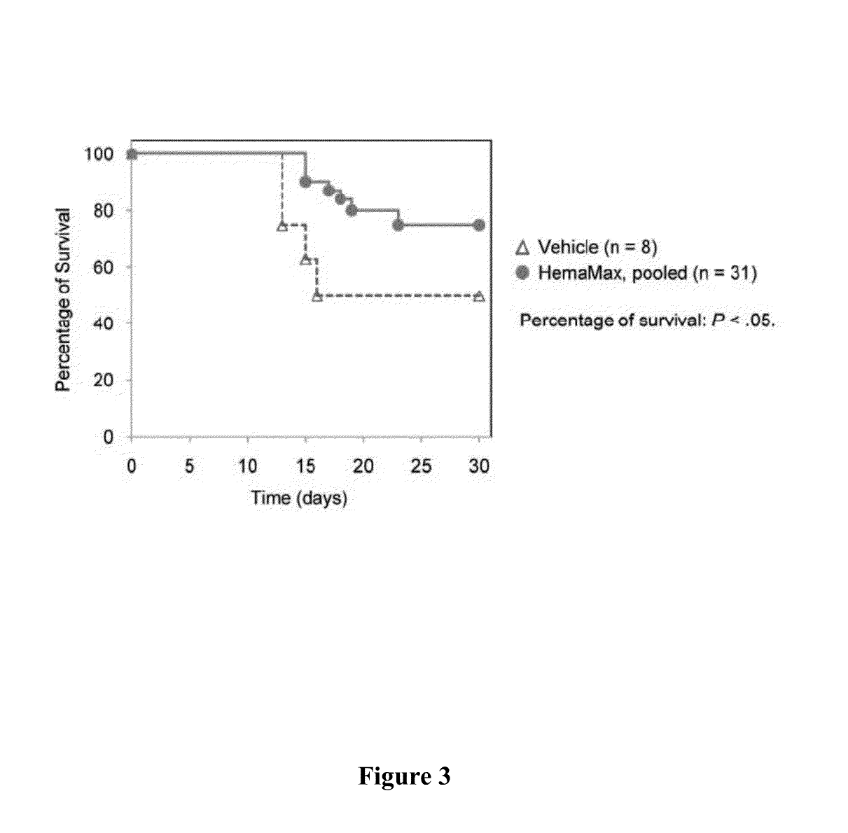

Radiomitigation study in myeloablated, lethally irradiated nonhuman primates (NHP demonstrated that HemaMax (rHuIL-12) increases survival, attenuates the platelet and WBC nadirs, and reduces the need for platelet transfusions in NHP. By way of example, FIG. 3 describes Kaplan-Meier Survival Curves of Irradiated, Unsupported Monkeys Treated with HemaMax™. Pooled HemaMax™ dosing group is shown. No antibiotics were used during the study.

### Example 4

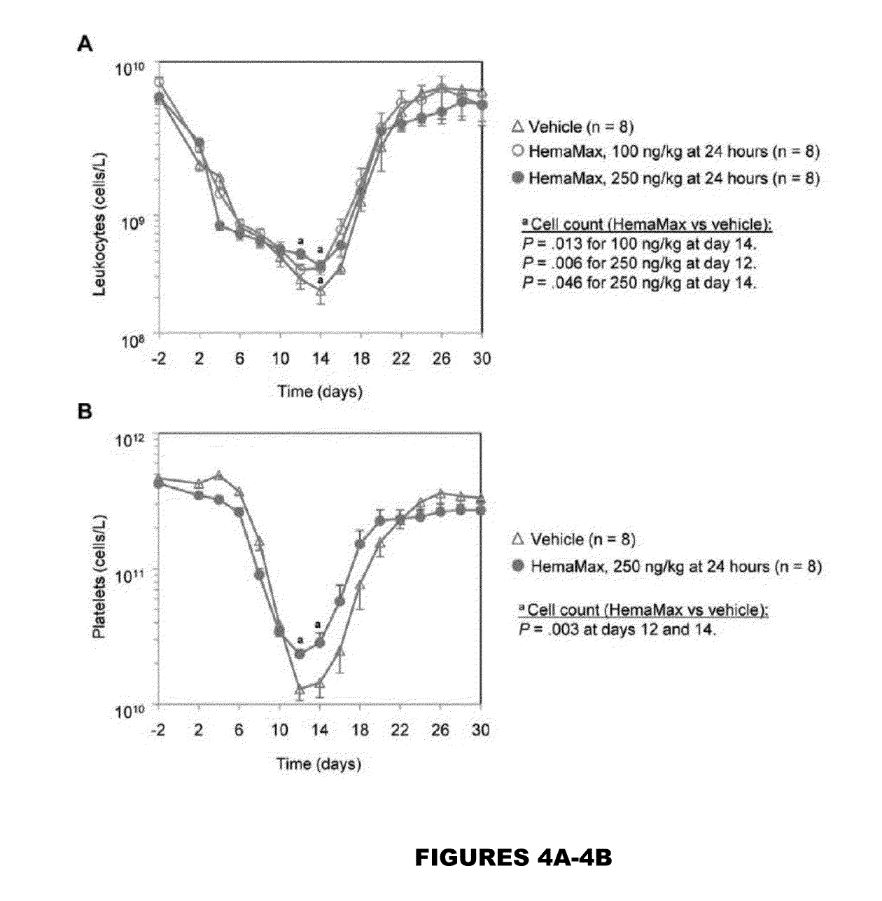

FIG. 4 describes Thrombocyte (a) and Leukocyte (b) Counts in Irradiated, Unsupported Rhesus Monkeys Treated With HemaMax™. Three analyses were conducted to assess differences in blood cell counts during the study period. In the first analysis, where blood cell counts were analyzed from day 1 up to day 30, animals treated with HemaMax™ had significantly higher numbers of leukocytes and thrombocytes at days 12 and 14 for the 100 ng/kg and 250 ng/kg doses of HemaMax™ as compared to animals treated with vehicle. Notably, lethal radiation NHP study, 80% of vehicle-treated monkeys would have required a platelet transfusion, whereas only 25% of HemaMax-treated monkey would have required a platelet transfusion.

### Example 5

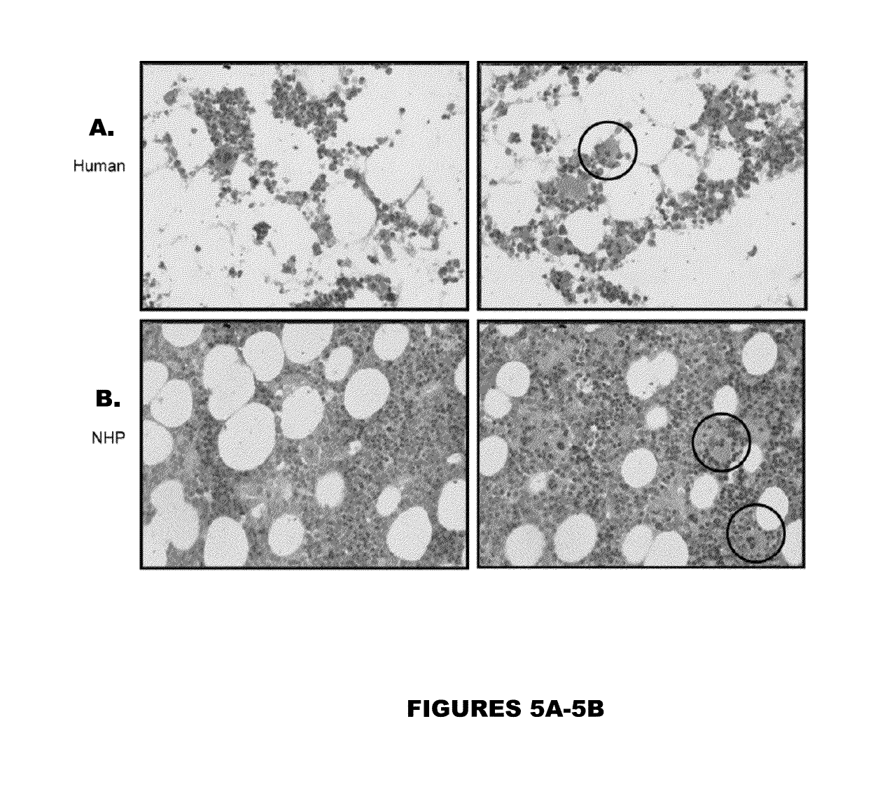

The role of the IL-12 receptor in megakaryopoiesis: The IL-12 receptor is found on human, NHP and mouse bone marrow progenitor cells and megakaryocytes. As shown in FIG. 5, tissues from human (a) and NHP (b) femoral bone marrow were immunohistochemically stained for IL-12Rβ2. Progenitor cells and megakaryocytes expressing IL-12Rβ2 are shown.

### Example 6

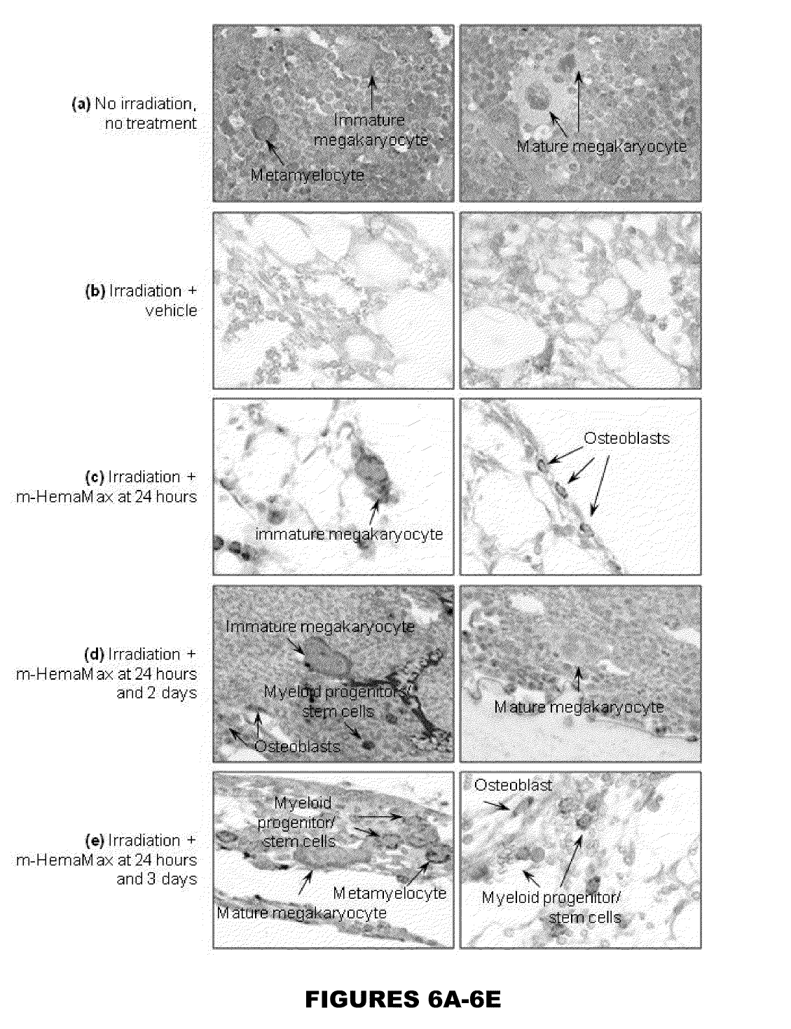

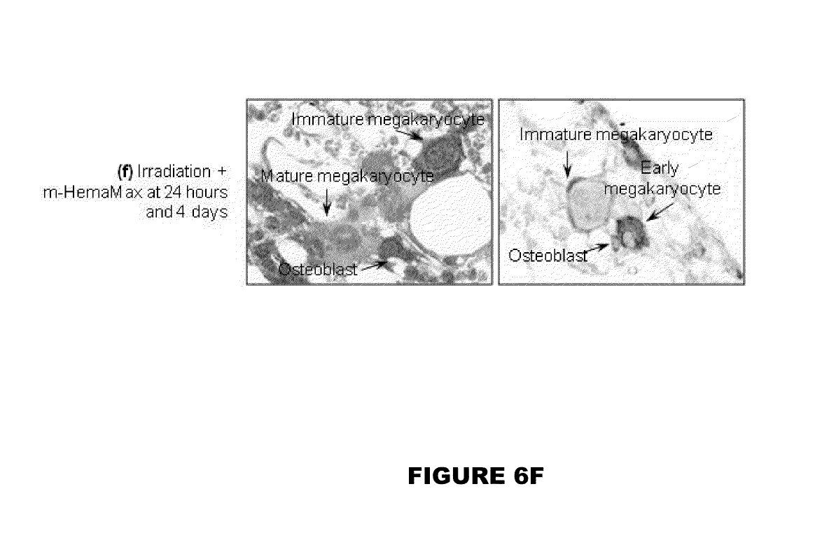

By way of example, FIG. 6 describes Murine IL-12 Promotes Hematopoietic Recovery in Irradiated Mice. Representative sections of femoral bone marrow from non-irradiated, untreated mice that were stained for IL-12Rβ2 (orange color) are shown in (a). Animals were subjected to TBI (8.0 Gy) and subsequently received vehicle or rMuIL-12 (20 ng/mouse) subcutaneously at the indicated times post irradiation (b-f). Femoral bone marrow was immunohistochemically stained for IL-12Rβ2 (orange color) 12 days after irradiation. While bone marrow from mice treated with vehicle lacked IL-12Rβ2-expressing cells and showed no signs of hematopoietic regeneration (b), mice treated with rMuIL-12 showed hematopoietic reconstitution and the presence of IL-12Rβ2-expressing megakaryocytes, myeloid progenitors, and osteoblasts (c-f).

### Example 7

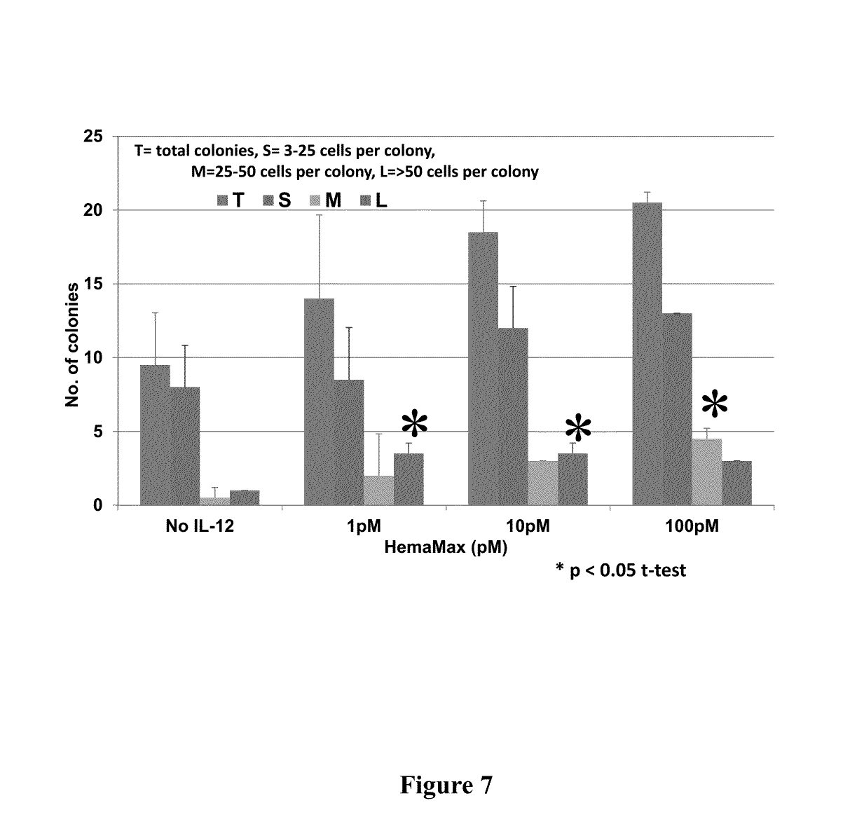

By way of example, FIG. 7 describes Lin-IL-12RB2-cells were plated in megacult medium at 5000 cells/slide with and without IL-12. Cultures stimulated with IL-12 resulted in larger colonies in comparison to cultures with media alone.

### Example 8

### Direct and Indirect Antitumor Effects of IL-12

HemaMax can be an anti-cancer agent, but it can also act as a hematopoietic adjuvant in cancer chemotherapy. The ability of HemaMax to act as an anti-tumor agent is also an additional benefit that can make HemaMax the preferred supportive care drug to address cytopenias induced by cancer treatments.

HemaMax works additively or synergistically with the exemplary chemotherapy regimens to reduce tumor size and recurrence over and above chemotherapy alone.

It is important to have a drug that will not have the counterproductive property of proliferating tumor growth when used to support hematopoietic recovery in cancer patients undergoing radiation or chemotherapy. We have already demonstrated that rMuIL-12 does not stimulate tumor growth in lymphoma and lung cancer murine models following radiation or chemotherapy. In fact, we have shown that murine IL-12 provides synergistic anti-tumor responses in both tumor models (Basile et al, 2008). In our studies, we observed success in obtaining significant decreases in tumor growth with murine IL-12, as compared to the use of the primary therapy alone (Basile et al, 2008). Many studies evaluated the anti-tumor role of IL-12 in different malignancies because of its pleiotropic biological activity (Cocco et al, 2009; Pistoia et al, 2009; Trinchieri, 2003). The mechanism of IL-12 anti-tumor activity includes at least i) immunostimulatory, ii) antiangiogenic, and iii) direct anti-tumor effects.

Anti-Thrombocytopenic Effect of rMuIL-12 in Murine Models of CIT

Effect of rMuIL-12 on CIT

The effect of rMuIL-12 administration in conjunction with gemcitabine/carboplatin chemotherapy (GC) (6.6 mg gemcitabine dose+2 mg carboplatin) was examined. Mice received GC intravenously on day 0. Subcutaneous murine IL-12 administration took place at one of the following three schedules and was compared to a control group, which did not receive murine IL-12:

Pre-dosing: rMuIL-12 was given 24 hours before chemotherapy treatment (n=18)

Post-dosing: rMuIL-12 was administered 48 hours after chemotherapy treatment (n=19) Pre/post dosing: rMuIL-12 was given 24 hours before chemotherapy and 48 hours after chemotherapy (n=13)

No rMuIL-12 (n=20)

Averages of % platelet counts from baseline and standard deviations for each group at day 8 were:

Pre-dosing group: 79±34%

Post-dosing group: 110±37%

Pre/Post dosing group: 111±33%

No rMuIL-12: 71±41%

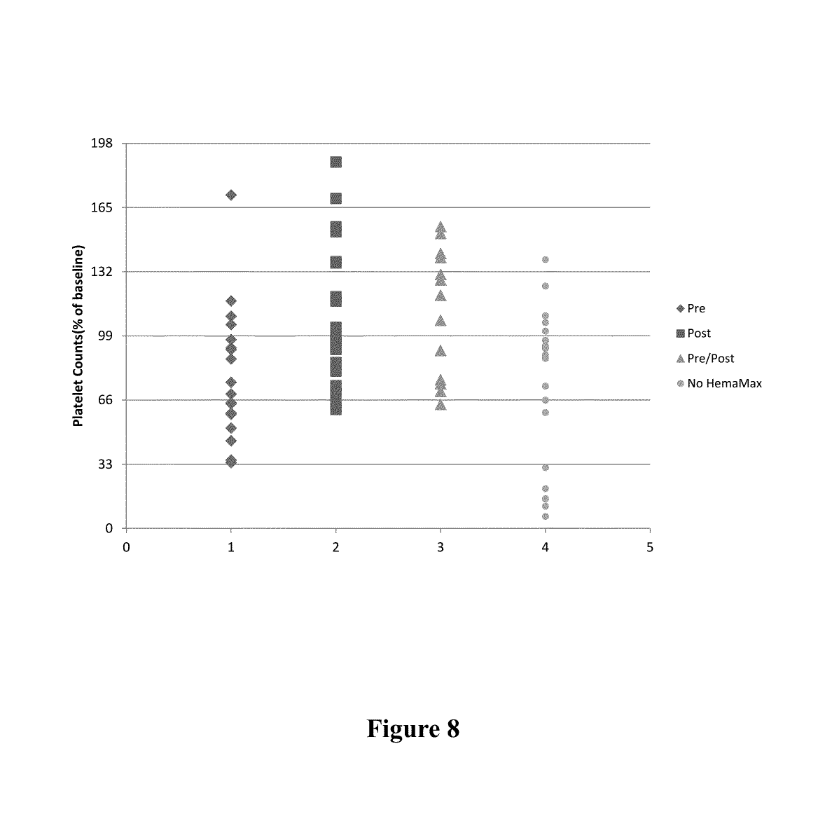

By way of example, FIG. 8 showed the Percentage of Individual Platelet Counts Compared to Baseline in CIT Mice Treated with rMuIL-12. None of the rMuIL-12 treated groups had an animal with a decrease in platelets of more than 33% (0 out of 50). In contrast the control group which did not receive rMuIL-12 had 6 mice out of 20 (30%) in which platelet levels compared to basal dropped to 33% or less.

### Example 9

### rMuIL-12 Effect on Tumor Growth in Lewis Lung Mouse Tumor Model with Concomitant GC Chemotherapy

The effect of rMuIL-12 administration on tumor growth in a mouse tumor chemotherapy model was evaluated. The tumor model used Lewis Lung tumor cells that were subcutaneously injected (100,000 cells) in each mouse. At a given time after tumor inoculation, mice were treated with chemotherapy. The gemcitabine/carboplatin chemotherapy regimen as described above was used and administered IP. rMuIL-12 was administered subcutaneously (20 ng) before, after, or before/after chemotherapy as indicated below.

Pre-dosing (n=16): rMuIL-12 was given 24 hours before chemotherapy treatment,

Post-dosing (n=16): rMuIL-12 was administered 48 hours after chemotherapy treatment

Pre/post dosing (n=16): rMuIL-12 was given 24 hours before chemotherapy and 48 hours after chemotherapy. Control (n=16)

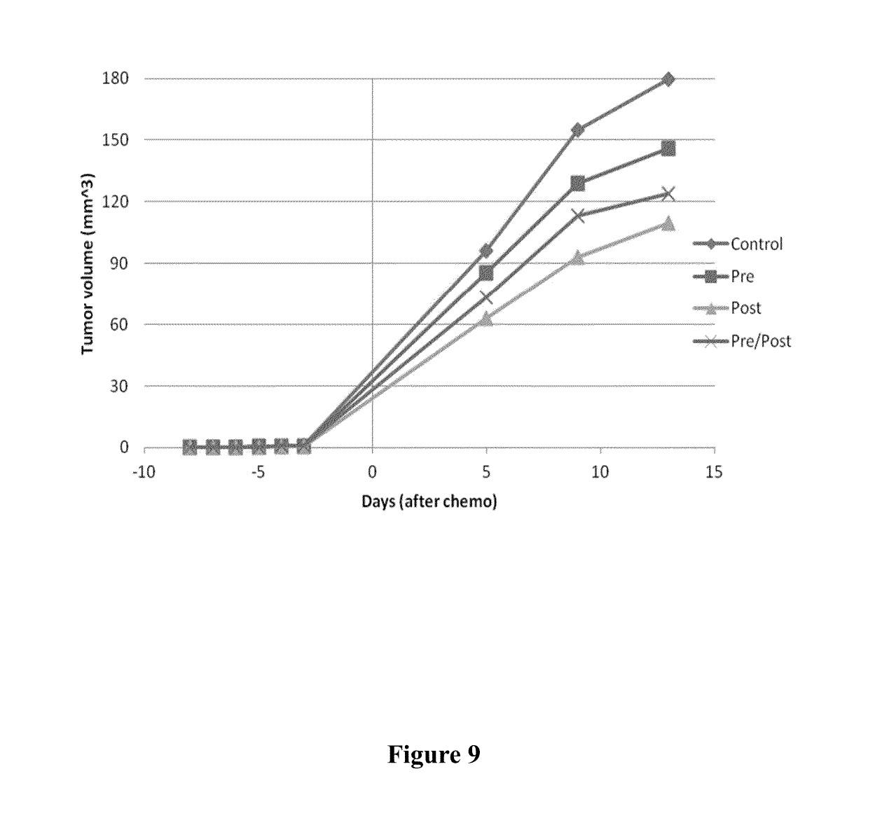

Tumor volume was monitored over time to compared tumor growth in the control compared to rMuIL-12 treated mice. As presented in FIG. 9, rMuIL-12 did not increase tumor growth in mice with growing Lewis Lung tumor cells under treatment with GC. The differences among groups in tumor growth were not statistically significant. The results indicate that a decrease in tumor growth with the addition of rMuIL-12 at any schedule is a trend.

By way of example, FIG. 9 showed that Lewis Lung Tumor Volume of Mice Under Therapy with GC Receiving rMuIL-12. Note: Time zero represents the time of chemotherapy administration. Tumor inoculation took place 11 days prior to chemotherapy administration.

### Example 10

Effect of rMuIL-12 with two rounds of gemcitabine monotherapy on tumor growth.

The study evaluate the effect of rMuIL-12 on tumor growth in a mouse chemotherapy model was followed up with a second study to examine the growth in the same Lewis Lung model when gemcitabine was used as monotherapy and administered two times, one week apart. rMuIL-12 was also administered as pre-dosing and post-dosing, as described above, and also with a 24 hour post group and on the same day as chemotherapy administration. rMuIL-12 was administered at either both weeks of chemotherapy or only the first week. There were five groups with n=5 for each group. These groups are described below: Chemotherapy once weekly: Gemcitabine only, administered IP, 6.6 mg rMuIL-12 given weekly, three schedules: 24 hours pre-chemotherapy (Pre); Same day as chemotherapy (SD); 24 hours post chemotherapy (Post1=24 hrs-post; Post2=48 hrs-post).

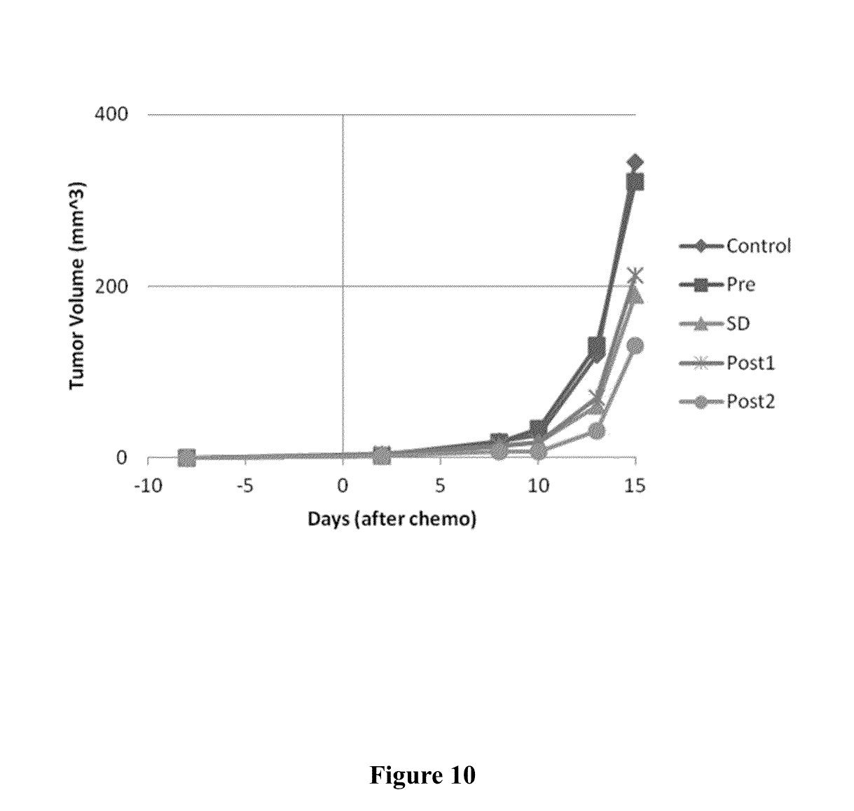

By way of example, FIG. 10 describes Lewis Lung Tumor Volume of Mice Under Monotherapy With Gemcitabine Receiving rMuIL-12. Day 0 is the time of the first chemotherapy. The second chemotherapy administration took place at day 7. Tumor inoculation took place on day 8. rMuIL-12 did not increase the tumor growth rate in this model system using only gemcitabine as the chemotherapy agent. The differences in growth among groups were not statistically significant. With the exception of the pre-dosing group, a trend for decreased tumor size was observed with rMuIL-12 administration.

### Example II

### Summary of Murine Data

Clinically relevant, allometrically determined doses of gemcitabine and carboplatin caused a reduction in platelet levels in 30% of mice consistent with the levels in humans that would necessitate dose reduction or delay, according to indicated dosage reduction guidelines, at 8 days after intravenous administration of chemotherapy. Administration of rMuIL-12 prior to, after or both prior to and after chemotherapy administration abrogated this decrease in platelet levels so as 0% of mice had platelet levels that dropped one-third of baseline level or more. Murine IL-12 did not increase tumor growth in murine lung cancer/gemcitabine regimen models at any schedule of rMuIL-12 administration relative to chemotherapy (prior to, post, or same day <6 hours after). Statistically non-significant trends were observed wherein murine IL-12 treatment decreased tumor growth in this model at any schedule of administration relative to chemotherapy.

### Example 12

### Summary and Design of an Exemplary First-In-Human (FIH) Clinical Study

The study, entitled A Phase 1, Double Blind, Placebo-Controlled, Single Ascending Dose Study of the Safety, Tolerability, Pharmacokinetics, and Pharmacodynamics of HemaMax™ (rHuIL-12) in Healthy Adult Volunteers (IND 104,091), is designed to determine the safety and tolerability with secondary objectives to evaluate the pharmacokinetics and immunogenicity of single ascending subcutaneous (SC) dose of HemaMax™ in healthy adult subjects.

Up to 30 healthy male and female adult subjects aged 18-45 years are enrolled in 4 consecutive cohorts of 6 subjects each, and sentinel subjects are used at each dose level. A single SC injection of investigational product (either HemaMax™ or placebo) is administered in the abdomen at dose levels of 2, 5, 10, and 20 μg. Cohorts of 6 subjects is evaluated per dose level (n=2 for placebo and n=4 for HemaMax™) in double-blind fashion. An additional expansion cohort of n=6 subjects (2 placebo and 4 HemaMax™) may be enrolled to receive the highest dose (or placebo) administered in the ascending dose portion of the study.

The first 2 subjects in each dose cohort are randomized to receive placebo or HemaMax™ at a ratio of 1:1 as a single SC injection in the abdomen. If the first 2 sentinel subjects (first group; group a) in a cohort do not present with any DLTs as assessed by the investigator and/or the Data Safety Committee (DSC) following a 7-day observation period, the next 4 subjects (second group; group b) are randomized to receive either placebo or HemaMax™ (1:3 placebo and HemaMax™). If a DLT is observed (as assessed by the investigator and/or the DSC), that subject are unblinded to the DSC. If the subject received HemaMax™, all dosing will cease and all treated subjects are followed as appropriate. If the unblinded subject was administered placebo, dosing will continue as planned. Following an in-patient observation period of at least 28 days from when the last subject in the cohort is dosed, all available cohort data (i.e., exposure and safety, including adverse events, vital signs, electrocardiogram, antigenicity and laboratory results) are reviewed by the DSC, and the DSC will decide if it is safe to ascend to the next dose level. Blood samples for antigenicity testing are collected at baseline as well as the study day 28, and the antigenicity test results are reviewed prior to escalation decision.

The goal of the Phase I project is to investigate and optimize HemaMax treatment schedule(s) when administered in combination with various chemotherapy regimens as to maximize efficacy and promote patient compliance. The optimization of the schedule of administration of HemaMax relative to chemotherapy administration is key to achieving clinical success.

Phase 1 Approach/Phase 1 Objectives

| | | Est. | | | Analytical |
|---|---|---|---|---|---|
| No. | Objective (Specific Aim) | Duration | n | Schedule | Methods |
| 1 | Evaluation and determination | 8 weeks | 108 male | GCB: | CBC, CFU-Meg |
| | of IL-12 treatment schedule(s) | | mice = 6 | 1X/wk (d 1 of | assay, & IHC |
| | for achieving optimal safety | | groups; 18 | wk for 8 wks); | |
| | and efficacy in a murine model | | mice/groups | IL-12 1X/8 wk | |
| | receiving intravenous | | | (on d 1, d 2, | |
| | gemcitabine (GCB) | | | d 3, or d 4) | |
| 2 | Evaluation and determination | 8 weeks | 108 male | GC/CPLB | CBC, CFU-Meg |
| | of IL-12 treatment schedule(s) | | mice = 6 | 1x/wk (d 1 of | assay, & IHC |
| | for achieving optimal safety | | groups; 18 | wk for 8 wks); | |
| | and efficacy in a murine model | | mice/groups | IL-12: 1X/8 wk | |
| | receiving intravenous combination | | | (on d 1, d 2, | |
| | therapy, gemcitabine/carboplatin | | | d 3, or d 4) | |
| | (GCB/CPL) | | | | |
| 3 | Safety and efficacy evaluation | 1 month | 30 male | TBD | CBC, CFU-Meg |
| | of optimal IL-12 schedules in | | mice; | | assay IHC, & |
| | murine tumor models receiving | | grouping | | tumor volume |
| | GCB only OR GCB/CPL | | TBD | | |

Time-Of-Administration (ToA)-Finding Studies (Phase 1, Objectives 1 & 2)

The Phase I project is intended to investigate and ultimately determine the optimal IL-12 treatment regimen in order to maximize safety and efficacy as applied to the condition of chemotherapy-induced thrombocytopenia (CIT). The Phase 1 research utilizes a healthy male and female murine models receiving weekly intravenous (IV) doses of gemcitabine (GCB) or a combination therapy of gemcitabine/carboplatin (GCB/CPL) over an eight-week period. Animals are treated with IL-12 on day 1 (d1), d2, d3, and/or d4. To assess efficacy (hematopoietic effect) and optimal treatment, analytical methods include complete blood cell count (CBC), colony-forming unit-megakaryocyte (CFU-Meg) assay, and immunohistochemistry (IHC). Successful study outcomes include the following: an increase in peripheral blood cell counts, specifically platelets, neutrophils and red blood cells, in treated groups versus vehicle group (analytical method: CBC); a thrombopoietic effect in treated groups versus vehicle group (analytical method: CBC; CFU-Meg assay; and IHC); a bone marrow-protective effect in treated groups versus vehicle group (analytical method: CFU-Meg assay and IHC); and determination of optimal IL-12 treatment regimen(s), which maximizes safety and efficacy as applied to the condition of chemotherapy-induced thrombocytopenia (CIT).

### Example 13

By way of example, FIG. 11 described Treatment Schedule which is directed to the Time-of-Administration (ToA)-Finding Study (Phase 1 Objective I). Evaluation and determination of IL-12 treatment schedule(s) for achieving optimal safety and efficacy (thrombopoiesis simulation) in a murine model receiving intravenous gemcitabine (GCB) (n=108).

### Example 14

By way of example, FIG. 12 showed the Treatment Schedule and Time-of-Administration (ToA)-Finding Study (Phase 1 Objective 2). Evaluation and determination of IL-12 treatment schedule(s) for achieving optimal safety and efficacy (thrombopoiesis simulation) in a murine model receiving intravenous gemcitabine (GCB) (n=108).

(Phase 1, Objective 3)

Objective 3 of the Phase 1 research utilizes the optimal treatment regimen(s) determined in Objective 1 and 2 of the Phase 1 research. Thus, the specific treatment schedule to be used in Objective 3 is yet to be determined. This study utilizes a murine tumor model receiving weekly intravenous (IV) doses of gemcitabine (GCB) or a combination therapy of gemcitabine/carboplatin (GCB/CPL) over 14-30 day period. The tumor model utilized will the Lewis Lung cancer solid tumor model. Again, animals are treated with IL-12 according to the optimal treatment schedule determined in Objectives 1 and 2. To assess efficacy (hematopoietic and antitumor effects) and optimal treatment, analytical methods include complete blood cell count (CBC) and measurements of tumor volumes. Successful study outcomes include the following: an increase in peripheral blood cell counts, specifically platelets, neutrophils and red blood cells, in treated groups versus vehicle group (analytical method: CBC); an observed antitumor response (analytical method: tumor volume); and determination of optimal IL-12 dosing regimen(s), which maximizes safety and efficacy as applied to the condition of chemotherapy-induced thrombocytopenia (CIT).

### Example 15

By way of example, FIG. 13 showed the Treatment Schedule in the POC Study (Phase 1 Objective 3). Safety and efficacy evaluation of optimal IL-12 schedules in murine tumor models receiving GCB only OR GCB/CPL (n=30).

### Example 16

### Phase 2

Phase 2 Objectives

| | | Est. | | | Analytical |
|---|---|---|---|---|---|
| No. | Objective (Specific Aim) | Duration | n | Schedule | Methods |
| 1 | HemaMax-Thrombopoiesis Computer | 6 months | n/a | n/a | algorithm |
| | Model: Algorithm-based investigation | | | | technology |
| | and confirmation of IL-12 treatment | | | | |
| | schedules for assessment of safety | | | | |
| | and efficacy | | | | |
| 2 | Phase 1b study to evaluate safety, | 18 months | 20 | See | CBC. |
| | tolerability, PK, and efficacy of | | | Methodology | Mesoscale |
| | HemaMax in combination with gemcitabine | | | Below | or ELISA |
| | in subjects with solid tumors | | | | |

HemaMax-Thrombopoiesis Computer Model (Phase 2, Objective 1)

The computer model consists of a comprehensive and very detailed mathematical description of thrombopoiesis, from the level of stem cells, to the level of platelets in the blood. The precise description of the model's many equations is beyond the scope of this proposal, but its general structure is based on the work of Skomorovski et al (PMID 14616973). The model is comprised of compartments comprehensively describing thrombopoiesis, including nine different stages of bone marrow cell progression, mature platelets, neutrophils and erythrocytes in peripheral blood, IL-12 and TPO concentration in the blood. Each compartment is further sub-divided according to maturation stage (age) of the cells in it. Additionally, the model can and is adjusted to model in conjunction with chemotherapeutic agents. This modeling effort provide a predictive model for human dose scheduling and further validate the schedule modeling developed in the Phase I project's preclinical murine work.

Phase 1b study evaluates safety, tolerability, PK, and efficacy of HemaMax in combination with gemcitabine in subjects with solid tumors (Phase 2, Objective 2)

Objectives

The primary objectives of this study are to evaluate the safety and tolerability of HemaMax in combination with gemcitabine for patients with solid tumors, and to establish the recommended phase 2 dose of HemaMax in combination with gemcitabine. The secondary objectives are to 1) characterize the PK parameters of HemaMax after first and second administration in combination with gemcitabine, 2) characterize the hematologic effects of HemaMax in patients receiving gemcitabine, specifically on blood counts and transfusion requirements, and 3) explore the clinical activity of HemaMax in combination with gemcitabine.

Methodology

| | |
|---|---|
| Test Product Dose, | HemaMax ™ (rHuIL-12) Drug Product is supplied in vials containing 0.65 |
| and Mode of | ml of 20 μg/mL HemaMax protein in 10 mM sodium phosphate, 150 mM |
| Administration: | sodium chloride, pH 6.0 with 0.1% (w/v) Poloxamer 188. The drug is |
| | given subcutaneously. The planned does levels are 50, 10, 200, and 300 |
| | ng/kg/dose. |
| Duration of | Cycle 1 consists of once a week treatment with both gemcitabine and |
| Treatment: | HemaMax ™ for 7 consecutive weeks, followed by 1 week of rest. Cycles |
| | 2 and beyond will consist of weekly treatment with both gemcitabine and |
| | HemaMax ™ for 3 consecutive weeks, followed by 1 week of rest. |
| | Patients will remain on treatment until one of the treatment |
| | discontinuation criteria are met. |
| Criteria for | All patients who receive any amount of HemaMax ™ are considered |
| Evaluation: | evaluable for safety. Patients who receive three planned does of both |
| | gemcitabine and HemaMax ™ during cycle 1 (days 1, 8, and 15) are |
| | considered evaluable for dose escalation evaluation. If a patient |
| | experiences a DLT during cycle 1 but does not receive all cycle 1 planned |
| | doses, that information is used in the dose escalation decision; the patient |
| | will not have to be replaced. |

This is a Phase 1b open-label, single-arm, dose-escalation, non-randomized study designed to evaluate the safety and tolerability of HemaMax™ dosed in combination with intravenous gemcitabine in subjects with solid tumors. HemaMax is being evaluated as an adjunctive supportive care medication (for CIT) in patients receiving gemcitabine, and thus an MTD for HemaMax isn't planned to be determined. Rather, four pre-defined dose levels are explored, the highest of which is expected to offer the benefit of protecting against CIT without any added toxicity. A standard ‘3+3’ dose escalation design is employed in this study. At least 3 patients are treated in each cohort, and up to 6 patients, depending on the presence or absence of DLT's. The dose of gemcitabine will remain constant at 1000 mg/m² (unless dose reductions are necessary due to toxicity), and will not be escalated. The first 3 patients in each cohort are treated for 21 days (3 doses of gemcitabine and 3 doses of HemaMax) before dose escalation. Dose escalation will proceed according as follows. If 0/3 patients at a dose level experience a DLT, dose escalation will continue to the next dose level. If 1/3 patients experiences a DLT, the cohort will expand to 6 patients. In a 6-patient cohort, if ≦1/6 patients experiences a DLT, dose escalation will continue to the next dose level. If >1/6 patients experiences a DLT, that dose level is considered above the MTD, and the previous dose level is expanded to 6 patients, unless 6 patients were already treated at the previous dose level. If there are no DLTs observed, six patients are treated at the highest planned dose of HemaMax to confirm the tolerability of that dose. Intravenous gemcitabine is infused over 30 minutes on days 1, 8, 15, 22, 29, 36, and 43 for the first cycle. In subsequent cycles, gemcitabine is infused over 30 minutes on days 1, 8, and 15 of each 28-day cycle. HemaMax is administered subcutaneously on days 1, 8, 15, 22, 29, 36, and 43 for the first cycle, 3 hours after the end of the gemcitabine infusion to minimize any potential drug-drug interaction. In cycle #2 and beyond, HemaMax is administered subcutaneously on days 1, 8, and 15, 3 hours after the end of each gemcitabine infusion. If a gemcitabine infusion is held for any reason (toxicity, disease progression, investigator discretion, or other reasons), HemaMax™ will also be held for that corresponding day/dose. Thus, HemaMax™ will only be administered on days in which subjects receive gemcitabine infusions. If gemcitabine infusions are delayed or given on a day that differs from the pre-specified study schedule, HemaMax™ will also be administered that day, 3 hours after the end of the gemcitabine infusion. Although serious adverse events (SAEs) specifically attributed to HemaMax are not expected, subjects enrolled in this trial of HemaMax is dosed in a deliberate and prudent manner at a facility fully equipped to manage AEs that may occur. Patients are closely observed on the day of dosing, and is seen frequently to monitor for toxicity. The first 3 patients treated at each dose level is observed for 21 days prior to dosing subsequent patients within that cohort. If any grade 3-4 toxicity develops after the first 21 days of treatment that is at least partially attributable to HemaMax™, though not considered a DLT technically, this information may be used at the investigator's discretion to 1) halt dose escalation of HemaMax™ in future patients/cohorts, and/or 2) modify the dose or discontinue HemaMax in other patients who have already started treatment. The Institutional Review Board (IRB) approved Subject informed Consent Form provide a fair and balanced perspective on the potential risks of HemaMax's administration. See Human Subjects section for further information, such as inclusion and exclusion criteria.

### Example 17

### Exemplary General Clinical Trial Design for Evaluating Efficacy

Objectives and Endpoints

TABLE 3

| | Objectives | | Endpoints | |
|---|---|---|---|---|
| Primary | 1. | To determine the safety, | 1. | Adverse endpoints AEs, and |
| | | tolerability, and recommended | | changes in laboratory values |
| | | weekly dose of HemaMax ™, in | | and vital signs |
| | | combination with gemcitabine for | | |
| | | subjects with solid tumors | | |
| Secondary | 1. | To characterize the steady-state | 1. | HemaMax ™ and gemcitabine PK |
| | | PK of HemaMax ™ and gemcitabine | | parameters following single dose |
| | 2. | To explore the clinical activity | | administration of HemaMax ™ and |
| | | of HemaMax ™ and gemcitabine in | | gemcitabine, including |
| | | subjects with solid tumors | | AUC₀₋ₜₐᵤ, AUC_{0-∞}, Cₘₐₓ, |
| | 3. | To characterize the effects of | | tₘₐₓ, and t_{1/2} (data permitting) |
| | | HemaMax ™ on hematological | 2. | Tumor response as assessed |
| | | parameters | | be RECIST 1.1. |
| | | | 3. | Frequency of transfusion, |
| | | | | rate of dosing delays due to |
| | | | | cytopenias, rates of Gr 3-4 |
| | | | | cytopenias |

Study Design/Schematic

This is a Phase I open-label, single-arm, dose-escalation, non-randomized study designed to evaluate the safety and efficacy of HemaMax™ dosed in combination with intravenous gemcitabine in subjects with solid tumors. HemaMax is evaluated as an adjunctive supportive care medication in patients receiving gemcitabine, and thus an MTD for HemaMax™ will not be determined. Rather, four pre-defined dose levels are explored, which are expected to offer the benefit of protecting against CIT without any added toxicity based on pre-clinical and FIH data. Three to six patients are treated per dose level, according to the presence or absence of DLTs (see section 3.4). If there are no DLTs observed, six patients are treated at the highest planned dose of HemaMax™ to confirm the tolerability of that dose.

Exemplary Treatment Schedule

Intravenous gemcitabine will be infused over 30 minutes on days 1, 8, 15, 22, 29, 36, and 43 for the first cycle. In subsequent cycles, gemcitabine will be infused over 30 minutes on days 1, 8, and 15 every 28 days. HemaMax™ will be administered subcutaneously on days 1, 8, 15, 22, 29, 36, and 43 for the first cycle, 3 hours after the end of the gemcitabine infusion. In Cycle #2, HemaMax™ will be administered subcutaneously on days 1, 8, and 15, 3 hours after the end of each gemcitabine infusion. HemaMax™ will not be administered in cycles #3 and beyond. If a gemcitabine infusion is held for any reason (toxicity, disease progression, investigator discretion, or other reasons), HemaMax™ will also be held for that corresponding day/dose. Thus, HemaMax™ will only be administered on days in which subjects receive gemcitabine infusions. If gemcitabine infusions are delayed or given on a day that differs from the pre-specified study schedule, if it is during cycle 1 or 2, HemaMax™ will also be administered that day, 3 hours after the end of the gemcitabine infusion.

The labeled dose of gemcitabine (1000 mg/m²) and schedule is not exceeded. The dose and schedule of gemcitabine can be decreased based on emerging tolerability data. The goal will be to define a dose and regimen that is well-tolerated and will provide adequate pharmacokinetics. This dose and regimen is the recommended phase 2 dose (R2PD).

Procedure for Dose Escalation

A standard ‘3+3’ dose escalation design is employed in this study. At least 3 patients are treated in each cohort, and up to 6 patients, depending on the presence or absence of DLT's. The dose of gemcitabine is constant at 1000 mg/m² (unless dose reductions are necessary due to toxicity), and is not escalated. Thus the “dose escalation” refers specifically to HemaMax™.

The first patient in each cohort is treated for 21 days (3 doses of gemcitabine and 3 doses of HemaMax™) before the next patient is treated at the same dose level.

There are four planned dose levels of HemaMax™ in this study as shown below:

TABLE 4

| Cohort # | HemaMax ™ dose |
|---|---|
| 1 | 50 ng/kg/dose |
| 2 | 100 ng/kg/dose |
| 3 | 200 ng/kg/dose |
| 4 | 300 ng/kg/dose |

Dose escalation is calculated according to the presence or absence of DLT's delineated in the table below. If 0/3 patients at a dose level experience a DLT, dose escalation continues to the next dose level. If 1/3 patients experiences a DLT, the cohort will expand to 6 patients. In a 6-patient cohort, if ≦1/6 patients experiences a DLT, dose escalation will continue to the next dose level. If >1/6 patients experiences a DLT, that dose level will be considered above the MTD, and the previous dose level will be expanded to 6 patients, unless 6 patients were already treated at the previous dose level. The recommended phase II dose (R2PD) is the highest dose level in this study at which ≦1/6 patients experiences a DLT.

TABLE 5

| Number of patients | |
|---|---|
| with a DLT in a | |
| given dose level | Dose escalation decision rule |
| 0 | Proceed to the next dose level. |
| 1 | Treat a total of 6 patients at this dose level. |

If ≦1/6 patients experiences a DLT, proceed
to the next dose level.
If >1/6 patients experiences a DLT, stop dose
escalation. Treat 3 additional patients in next
lower dose level, unless 6 patients already
treated at next lower dose level.

| | |
|---|---|
| 2 | Stop dose escalation. Treat 3 additional patients |
| | in next lower dose level unless 6 patients already |
| | treated at next lower dose level. |

DLT

A dose-limiting toxicity is defined as toxicity occurring during the first 21 days after administration of the first dose of gemcitabine and HemaMax™ that meets any of the following criteria: Grade 4 neutropenia lasting ≧5 days; Grade 3-4 febrile neutropenia; Grade 4 thrombocytopenia; Grade 3 or 4 non-hematologic toxicity (except electrolyte disturbances responsive to correction with 24 hours; or diarrhea/nausea and vomiting that responds to standard medical care); Treatment delay of 14 days or greater due to unresolved toxicity; A grade 2 or greater non-hematological toxicity that in the judgment of the investigator and medical monitor is dose-limiting; A 2-grade increase (grade 1 to grade 3, or grade 2 to grade 4) in AST or ALT for patients with baseline elevated AST or ALT.

MTD and R2PD

Depending on the frequency of DLTs, the MTD, if two or more subjects in any cohort experience a DLT, then by definition, the MTD has been exceeded. In this case, the MTD is the highest dose at which ≦one out of six subjects experiences a DLT during the first 21 days after the first administration of gemcitabine and HemaMax™.

If <1/6 patients at 300 ng/kg/dose of HemaMax™ experience a DLT, then there is by definition no MTD, and 300 ng/kg/dose will be considered the R2PD unless there is a lower dose that provides sufficient activity with a superior tolerability and safety profile.

Continued Treatment of Subjects Beyond the First Cycle

A subject can continue treatment with HemaMax™ and gemcitabine through cycle 2, and single agent gemcitabine thereafter, until the Treatment Discontinuation Criteria are met (see Section 3.8).

Intra-Subject Dose Escalation

Intra-subject dose escalation of HemaMax™ will not be allowed in this study. If a patient requires a dose reduction of gemcitabine, the dose can be re-escalated back to the standard dose of 1000 mg/m² at the investigator's discretion, in conjunction with discussion with the medical monitor.

Treatment Discontinuation Criteria

Each subject may continue to receive HemaMax™ and gemcitabine for cycle #2, and single agent gemcitabine thereafter, until one of the following occurs:

Disease progression based on RECIST 1.1. The investigator may discuss with the medical monitor continuing treatment in a subject who is receiving benefit but has met the criteria for disease progression.

Intercurrent illness that prevents further administration of study drug(s).

Adverse event that is considered by the investigator or medical monitor to warrant permanent discontinuation of study drug(s).

A clinically significant adverse event leading to an interruption of treatment for greater than 14 days. Subjects will be allowed up to a 14-day delay in dosing for toxicity to resolve or for scheduling difficulties. If the investigator and medical monitor conclude that continued treatment will benefit a subject who has had >14-day treatment delay, the subject may continue therapy with the approval of the medical monitor.

Subject withdraws consent for further treatment or data collection.

If the subject withdraws consent for further treatment, follow-up visits should continue.

If the subject withdraws consent for further treatment and data collection, then non additional study visits or data collection should occur.

Investigational Product Dosage/Administration

TABLE 6
Investigational product dosage/administration of HemaMax ™

| Product Name | HemaMax ™ |
|---|---|
| Formulation | The HemaMax ™ (rHuIL-12) Drug Product |
| description | vial contains 0.65 mL of 20 μg/mL rHuIL-12 |
| | protein in 10 mM sodium phosphate, 150 mM |
| | sodium chloride, pH 6.0 with 0.1% (w/v) |
| | Poloxamer 188 (withdrawal volume of 0.50 mL). |
| Dosage form | Vials |
| Unit dose | 20 μg/mL in 2 mL clear vials |
| strength(s)/dos- | |
| age levels | |
| Physical | Solution is clear and colorless |
| description | |
| Route/duration | Subcutaneously |
| Dosing | Subcutaneously HemaMax ™ is injected in |
| instructions | the abdomen on days 1, 8, 15, 22, 29, 36, |
| | and 43 during cycle 1; and on days 1, 8, |
| | and 15 during cycle 2. |

TABLE 7
Investigational product dosage/administration of gemcitabine

| Product name | Gemcitabine/Gemzar | |
|---|---|---|
| Formulation | Gemzar (gemcitabine HC1) is a nucleoside analogue | |
| description | that exhibits antitumor activity. Gemcitabine HC1 is | |
| | 2′-deoy-2′,2′-difluorocytidine monohydrochloride | |
| | (β-isomer). | |
| Dosage form | Vials | |
| Unit dosage | 200 mg white, lyophilized | 1 g white, lyophilized |
| strength(s)/dos- | powder in a 10-mL size | powder in a 50-mL size |
| age levels | sterile single vial | sterile single use vial |
| | (no. 7501) | (no. 7502) |
| | NDC 0002-7501-01 | NDC 0002-7502-01 |

| | |
|---|---|
| Physical | Gemcitabine HC1 is a white to off-white sold. It is a |
| description | soluble in water, slightly soluble in methanol, and |
| | practically insoluble in ethanol and polar organic |
| | solvents. The clinical formulation is supplied in a |
| | sterile form for an intravenous use only. |
| Route/duration | Intravenously |
| Dosing | Intravenous gemcitabine is infused over 30 minutes on |
| Instructions | days 1, 8, 15, 22, 29, 36, and 43 during cycle 1; and on |
| | days 1, 8, and 15 of subsequent cycles. NOTE: |
| | Prolongation of gemcitabine infusion time beyond 60 |
| | minutes has been shown to increase toxicity. |

Dose Modifications for Gemcitabine

Dose modifications for gemcitabine may be required in response to toxicity or for subjects who cannot tolerate full-dose gemcitabine. All toxicities will be graded based on the NCI CTCAE version 4.02. For all toxicities except ANC and platelet counts, discontinuation or interruption of dosing with gemcitabine may be considered when subjects develop ≧grade 2 toxicity. Gemcitabine can be restarted at the current dose level when the toxicity improves to grade 1 or less. If the toxicity recurs, then consider restarting gemcitabine with at least a 25% dose reduction. For those subjects who may not be able to tolerate the prescribed gemcitabine dose, dose modifications will be allowed per investigator clinical judgment and practice.

Recommendations for dose modifications based on decreases in ANC and/or platelet counts are in Table 9.

TABLE 9
Dose modifications for gemcitabine based on
decreases in ANC and/or platelet counts

| Hematology parameter | Gemcitabine dose modification |
|---|---|
| ANC ≧ 1000 × 10⁶/L and platelet | Administer 100% of full dose |
| count ≧ 100,000 × 10⁶/L | |
| ANC 500-999 × 10⁶/L or platelet | Administer 75% of full dose |
| count 50,000-99,9999 × 10⁶/L | |
| ANC < 500 × 10⁶/L or platelet | Hold dose |
| count < 50,000 × 10⁶/L | |

Procedures During Screening

Within 28 days prior to treatment:

Tumor measurement (RECIST): tumor markers, if applicable
The same assessment method used at baseline should be used consistently for all evaluations throughout the study

Informed consent

Baseline demographics

Medical history

Medication history

Within 72 hours prior to treatment:

Height & weight

Physical examination

ECOG Performance Status

Serum pregnancy test for women of childbearing potential
A negative serum pregnancy test must be documented

Inclusion/exclusion criteria review

Urinalysis

12-lead ECG

Hematology

Chemistry

Tumor markers, if applicable

Procedures During Treatment

Cycle 1, Day 1
Vital signs just prior to the start of the gemcitabine infusion, just prior to administration of HemaMax™, and 2 hours and 4 hours after the administration of HemaMax™
12-lead ECG just prior to administration of HemaMax™, and 8 hours after administration of HemaMax™
Administration of intravenous gemcitabine
Administration of subcutaneous HemaMax™
Blood sampling for PK studies (see Table 12 for details regarding PK timepoints)
Antigenicity and NAB testing
Adverse event assessment, if necessary

Cycle 1, Day 2

Vital signs

Hematology

Chemistry

Blood sampling for PK studies

12-lead ECG

Cycle 1, Day 8

Interim medical history

Medication history

Weight

Physical examination

Vital signs

ECOG Performance Status

Hematology

Chemistry

Urinalysis

Administration of intravenous gemcitabine

Administration of subcutaneous HemaMax™

Blood sampling for PK studies

Adverse event assessment

12-lead ECG just prior to administration of HemaMax™

Cycle 1, Day 15

Interim medical history

Medication history

Weight

Physical examination

Vital signs

ECOG Performance Status

Hematology

Chemistry

Urinalysis

Administration of intravenous gemcitabine

Administration of subcutaneous HemaMax™

Adverse event assessment

Cycle 1, Day 22

Interim medical history

Medication history

Weight

Physical examination

Vital signs

ECOG Performance Status

Hematology

Chemistry

Urinalysis

Administration of intravenous gemcitabine

Administration of subcutaneous HemaMax™

Adverse event assessment

Cycle I, Day 29

Interim medical history

Medication history

Weight

Physical examination

Vital signs

ECOG Performance Status

Hematology

Chemistry

Urinalysis

Administration of intravenous gemcitabine

Administration of subcutaneous HemaMax™

Adverse event assessment

Cycle 1, Day 36

Interim medical history

Medication history

Weight

Physical examination

Vital signs

ECOG Performance Status

Hematology

Chemistry

Urinalysis

Administration of intravenous gemcitabine

Administration of subcutaneous HemaMax™

Adverse event assessment

Cycle 1, Day 43

Interim medical history

Medication history

Weight

Physical examination

Vital signs

ECOG Performance Status

Hematology

Chemistry

Urinalysis

Antigenicity and NAB testing

Administration of intravenous gemcitabine

Administration of subcutaneous HemaMax™

Adverse event assessment

Cycle 1, Day 50

No assessments or procedures on this day

Cycle 2, Day 1

Interim medical history

Medication history

Weight

Physical examination

Vital signs

ECOG Performance Status

Hematology

Chemistry

Administration of intravenous gemcitabine

Administration of subcutaneous HemaMax™

Adverse event assessment

Cycle 2, Day 8

Interim medical history

Medication history

Weight

Physical examination

Vital signs

ECOG Performance Status

Hematology

Chemistry

Administration of intravenous gemcitabine

Administration of subcutaneous HemaMax™

Adverse event assessment

Cycle 2, Day 15

Interim medical history

Medication history

Weight

Physical examination

Vital signs

ECOG Performance Status

Hematology

Chemistry

Administration of intravenous gemcitabine

Administration of subcutaneous HemaMax™

Adverse event assessment

Cycle 2, Day 22

Interim medical history

Medication history

Weight

Physical examination

Vital signs

ECOG Performance Status

Hematology

Chemistry

Adverse event assessment

Cycle 3 and Thereafter, Day 1
Interim medical history
Tumor markers, if applicable
Medication history
Weight
Physical examination
Vital signs
ECOG Performance Status
Hematology
Chemistry
Administration of intravenous gemcitabine
Adverse event assessment
Disease assessment using RECIST (this will subsequently be performed on day 1 of odd-numbered cycles only, i.e. cycles 5, 7, 9, etc. . . . )

Cycle 3 and Thereafter, Day 8

Interim medical history

Medication history

Weight

Physical examination

Vital signs

ECOG Performance Status

Hematology

Chemistry

Administration of intravenous gemcitabine

Adverse event assessment

Cycle 3 and Thereafter, Day 15

Interim medical history

Medication history

Weight

Physical examination

Vital signs

ECOG Performance Status

Hematology

Chemistry

Administration of intravenous gemcitabine

Adverse event assessment

End of Treatment

When patients are taken off (lithe study, the following activities should be done:
Vital signs
Interim medical history
Physical examination
Adverse event assessment
Antigenicity and NAB testing
Hematology
Chemistry

If a patient is hospitalized or otherwise unable to come to the treatment center for the end of study visit, every effort should be made to have the patient return at some point for final antigenicity testing.

Pharmacokinetic Sampling Timetable

TABLE 12

| | | PK Blood | PK Blood |
|---|---|---|---|
| Cycle, | | sampling- | sampling- |
| Day | Time Point | HemaMax ™ | gemcitabine |
| Cycle 1, | Pre-gemcitabine infusion | | X |
| Day 1 | 30 minutes post- | | X |
| | gemcitabine infusion | | |
| | Pre-HemaMax ™ administration | X | X |
| | 1 hour post-HemaMax ™ | X | |
| | administration | | |
| | 2 hours post-HemaMax ™ | X | |
| | administration | | |
| | 4 hours post-HemaMax ™ | X | |
| | administration | | |
| | 8 hours post-HemaMax ™ | X | |
| | administration | | |
| Cycle 1, | 24 hours post-HemaMax ™ | X | X |
| Day 2 | administration | | |
| Cycle 1, | Pre-HemaMax ™ administration | X | X |
| Day 8 | | | |

The PK sampling in the above table should be done within 10 minutes of the designated timepoint. If this is not possible due to any unforeseen circumstance(s), the PK sample may still be drawn, with the actual time being clearly documented.

Data Analysis and Statistical Considerations

Hypotheses and Treatment Comparisons

The primary objective of this study is to determine the recommended Phase 2 dose based on available safety, PK and any clinical activity data of HemaMax™ when administered in combination with gemcitabine; thus, no formal statistical hypotheses will be tested. Analysis will be focused on comparison between dose levels and will be descriptive and exploratory.

Sample Size Assumptions

The sample size is not driven by statistical considerations. The total number of subjects will depend on the presence or absence of DLTs. However, the maximum anticipated number of subjects will be approximately 24.

Analysis of Study Endpoints

Primary Endpoint

The primary endpoint of this phase 1 study is to evaluate the safety, tolerability, and recommended phase 2 dose of HemaMax™ given weekly in combination with gemcitabine for subjects with solid tumors.

All patients who receive any amount of HemaMax™ are considered evaluable for safety. Patients who receive all four planned doses of both gemcitabine and HemaMax™ during cycle 1 are considered evaluable for dose escalation evaluation. If a patient experiences a DLT during cycle 1 but does not receive all cycle 1 planned doses, that information is used in the dose escalation decision; the patient is not to be replaced.

Secondary Endpoints

Secondary endpoints in this study include pharmacokinetic parameters, including, AUC₀₋ₜₐᵤ, AUC_{0-∞}, Cₘₐₓ, tₘₐₓ, and t_{1/2} (data permitting); response rates using RECIST 1.1; as well as frequency of transfusions, rate of dosing delays due to cytopenias, and rates of Gr 3-4 cytopenias.

Exploratory Endpoint

There is some evidence that serum haptoglobin levels may predict sensitivity to the hematologic toxicity of gemcitabine. Serum haptoglobin levels will be collected at baseline in this study to explore this concept, but will not be used to select patients, and will not be measured after patients have begun treatment to influence any treatment decisions (unless haptoglobin is drawn for another reason, such as to rule out hemolysis).

The instant disclosure also contemplates the use of the following exemplary primary and/or secondary endpoints and assessment criteria, including, for example, incidence of Adverse Events; Duration of Grade 3 or 4 Thrombocytopenia; the duration of grade 3 or 4 thrombocytopenia (e.g. platelet count <50×10̂9/L) experienced during the first on study chemotherapy cycle by treatment group; incidence of Participants Experiencing Grade 3 or 4 Thrombocytopenia (<50×10̂9/L) During First Treatment Cycle; participant Incidence of Platelet Transfusions; incidence of participants who were administered platelet transfusions during first on study treatment cycle; Platelet Count.

Physical Exams

A physical examination will be performed by a qualified physician or designee according to local practice.

Vital Signs

Vital sign measurements will include temperature, systolic and diastolic blood pressure and pulse rate. Height (baseline only) and weight will also be measured and recorded.

Electrocardiogram (ECG)

12-lead ECGs will be obtained during the study using an ECG machine (Mortara system) that automatically calculates the heart rate and measures PR, QRS, QT, and QTc intervals. At each assessment a 12-lead ECG will be performed by qualified personnel at the site after the subject has rested at least five minutes in a semi-recumbent or supine position.

If there are any clinically significant abnormalities including but not limited to a QTcF>450? msec, findings are confirmed with two additional ECGs taken at least 5 minutes apart.

Clinical Laboratory Assessments

Hematology, chemistry, urinalysis, and serum pregnancy tests as listed in will be analyzed at the site's laboratory. Haptoglobin may be sent out to a qualified laboratory and not done on-site.

Performance Status

The performance status assessment is based on the ECOG scale [Oken, 1982]:

0—Fully active, able to carry on all pre-disease performance without restriction.

1—Restricted in physically strenuous activity but ambulatory and able to carry out work of a light or sedentary nature (e.g., light house work, office work).

2—Ambulatory and capable of all self-care but unable to carry out any work activities. Up and about more than 50% of waking hours.

3—Capable of only limited self-care, confined to bed or chair more than 50% of waking hours.

4—Completely disabled. Cannot carry on any self-care. Totally confined to bed or chair.

Anti-Cancer Activity

Disease assessment will include imaging (e.g., computed tomography, magnetic resonance imaging, bone scan, plain radiograph), and physical examination (as indicated for palpable/superficial lesions).

For subjects whose disease may be followed by well-characterized tumor markers, disease assessments should include results of tumor marker measurements. Disease assessment will be completed within four weeks prior to the first dose of gemcitabine, then every eight weeks (±five days) thereafter, and at the final study visit.

It is not necessary to repeat radiologic assessments at the final study visit if the subject was withdrawn due to disease progression. Subjects whose disease responds (either complete response or partial response) should have a confirmatory disease assessment performed four weeks (±five days) after the date of the assessment during which response was demonstrated. More frequent disease assessments may be performed at the discretion of the investigator. To ensure comparability between baseline and subsequent assessments, the same method of assessment and the same technique will be used when assessing response.

Pharmacokinetics

Blood samples for pharmacokinetic analysis for both HemaMax™ and gemcitabine will be collected at the time points indicated in Table 12. The actual date and time of each blood sample collection will be recorded. The timing of PK samples may be altered and/or PK samples may be obtained at additional time points to ensure thorough PK monitoring.

PK Samples—HemaMax™

See Table 12 for PK timepoints. Collect the blood samples in Vacutainer® collection tubes containing spray-dried dipotassium ethylene diamine tetra-acetic acid (K2EDTA). Following collection, invert the samples 8 to 10 times to mix with the anticoagulant. Maintain the tubes in an ice/water bath until centrifugation. Within 60 minutes of collection, centrifuge samples at 4° C. for 10 minutes at approximately 1500G. Pipette approximately 2 mL of plasma into the appropriate aliquot tube. Maintain the tubes in an ice/water bath until freezing. Freeze the samples at approximately −70° C. within 60 minutes of centrifugation.

PK Samples—Gemcitabine

See Table 12 for PK timepoints. Collect the blood samples in Vacutainer® collection tubes containing sodium heparin (Na Hep) and treated with tetrahydrouridine (THU) (0.1 mg/mL in plasma) to inhibit cytidine deaminase, which metabolizes Gemcitabine to dFdU. Within 60 minutes of collection, centrifuge samples at 4° C. for 10 minutes at approximately 1500G. Pipette approximately 0.5 mL of plasma into the appropriate aliquot tube. Maintain the tubes in an ice/water bath until freezing. Freeze the samples at approximately −70° C. within 60 minutes of centrifugation.

Antigenicity and Neutralizing Antibody Testing

These samples will be collected on Cycle 1, Day 1 (pre-dose), on Cycle 1, Day 43, and at the end of study visit.

Collect the blood samples into Vacutainer® collection tubes containing spray-dried dipotassium ethylene diamine tetra-acetic acid (K₂EDTA). Following collection, invert the samples 8 to 10 times to mix with the anticoagulant, and maintain the tubes in an ice/water bath until centrifugation. Within 60 minutes of collection, centrifuge the samples at 4° C. for 10 minutes at approximately 1500G. Pipette the plasma into 4 appropriately labeled aliquots, each approximately 0.5 mL in volume. Maintain the tubes in an ice/water bath until freezing. Freeze the samples at approximately −70° C. within 60 minutes of centrifugation.

Concomitant Medications and Non-Drug Therapies

Permitted Medications

Palliative and supportive care for disease-related symptoms will be offered to all patients in the study. As such, patients may receive antibiotics, anti-emetics, anti-diarrheals, analgesics, and other care as deemed appropriate, and in accordance with institutional guidelines. Patients may not receive any other anticancer agent while on the study; however, patients on adjuvant anti-estrogen therapy for a coexisting diagnosis of hormone-sensitive breast cancer (not the malignancy for which the patient is on the current study) may remain on such therapies. Patients may also receive bisphosphonate treatment or denosumab for bone metastases while on the study.

Supportive care adjunctive therapies such as erythropoietin-stimulating agents (ESAs) and colony-stimulating factors may be administered per ASCO guidelines at the discretion of the investigator after completing the first 21 days on study. They may be given earlier if deemed medically necessary.

If a patient experiences any allergic or cytokine-mediated reaction (fever, chills, pruritus, hives, etc.) to HemaMax™, he/she may be premedicated prior to subsequent doses of HemaMax™. If more than one patient experiences a similar reaction, all subsequent patients will be placed on the same premedication regimen prior to all doses of HemaMax™. The suggested regimen is:

Dexamethasone 10 mg IV

Ranitidine 50 mg IV

Diphenhydramine 50 mg IV

Acetaminophen 650 mg PO

Non-Drug Therapies

Subjects abstain from using herbal preparations/medications throughout the study until the final study visit. These herbal medications include, but are not limited to, St. John's Wort, kava, ephedra (ma huang), gingko biloba, dehydroepiandrosterone (DHEA), yohimbe, saw palmetto, and ginseng. Standard doses of individual vitamins or multivitamins are permitted.

Investigational Product

Preparation/Handling/Storage/Accountability

Gemcitabine

The recommended diluent for reconstitution of Gemzar is 0.9% Sodium Chloride Injection without preservatives. Due to solubility considerations, the maximum concentration for Gemzar upon reconstitution is 40 mg/mL. Reconstitution at concentrations greater than 40 mg/mL may result in incomplete dissolution, and should be avoided. To reconstitute, add 5 mL of 0.9% Sodium Chloride Injection to the 200 mg vial or 25 mL of 0.9% Sodium Chloride Injection to the 1 g vial. Shake to dissolve.

These dilutions each yield a gemcitabine concentration of 38 mg/mL which includes accounting for the displacement volume of the lyophilized powder (0.26 mL for the 200 mg vial or 1.3 mL for the 1 g vial). The total volume upon reconstitution will be 5.26 mL or 26.3 mL, respectively. Complete withdrawal of the vial contents will provide 200 mg or 1 g of gemcitabine, respectively. The appropriate amount of drug may be administered as prepared or further diluted with 0.9% Sodium Chloride Injection to concentrations as low as 0.1 mg/mL. Reconstituted Gemzar is a clear, colorless to light straw-colored solution. After reconstitution with 0.9% Sodium Chloride Injection, the pH of the resulting solution lies in the range of 2.7 to 3.3. The solution should be inspected visually for particulate matter and discoloration, prior to administration, whenever solution or container permit. If particulate matter or discoloration is found, do not administer. When prepared as directed, Gemzar solutions are stable for 24 hours at controlled room temperature 20° C. to 25° C. (68° F. to 77° F.) [See USP]. Discard unused portion. Solutions of reconstituted Gemzar should not be refrigerated, as crystallization may occur. The compatibility of Gemzar with other drugs has not been studied. No incompatibilities have been observed with infusion bottles or polyvinyl chloride bags and administration sets. Unopened vials of Gemzar are stable until the expiration date indicated on the package when stored at controlled room temperature 20° C. to 25° C. (68° F. to 77° F.) [See USP]. Caution should be exercised in handling and preparing Gemzar solutions. The use of gloves is recommended. If Gemzar solution contacts the skin or mucosa, immediately wash the skin thoroughly with soap and water or rinse the mucosa with copious amounts of water. Although acute dermal irritation has not been observed in animal studies, two of three rabbits exhibited drug-related systemic toxicities (death, hypoactivity, nasal discharge, shallow breathing) due to dermal absorption. Procedures for proper handling and disposal of anti-cancer drugs should be followed.

HemaMax™

The pharmacist prepares the dosing solution in the form of a filled syringe for injection into the subject. In this trial, the Investigational Product (IP) consists of the HemaMax (rHuIL-12) Drug Product in 2 mL clear vials. The HemaMax (rHuIL-12) Drug Product vial contains 0.65 mL of 20 μg/mL rHuIL-12 protein in 10 mM sodium phosphate, 150 mM sodium chloride, pH 6.0 with 0.1% (w/v) Poloxamer 188 (withdrawal volume of 0.50 mL). These solutions are clear and colorless. Remove vials from refrigerator and allow to sit at room temperature for at least 15 minutes prior to preparation of doses. A BD syringe with polypropylene barrel with detached 25G ⅝ needle or BD Tuberculin Syringe (catalog #305553, 27 g ½ needle attached) has been shown to be compatible. The syringe with the prepared solution can be kept at room temperature for 6 hours. If a longer storage time is desired the syringe can be stored at 2-8° C. for 24 hours. If a syringe with separate needle is used, overfill the dose syringe by approximately 0.1 mL, then remove the needle and replace with new needle, and gently expel until the appropriate dose is reached.

5 μg HemaMax (rHuIL12)

Drug Product Dose Preparation

Requires:

1 vial of HemaMax (rHuIL-12) Drug Product, 1 vial of HemaMax Placebo, 2 BD Syringes and 3 BD 25G ⅝ needles
i. Withdraw 0.65 mL from the HemaMax (rHuIL-12) Drug Product vial.
ii. Mix solution via several inversions of the vial 3 to 4 times and then gently tap the bottom of the vial 3 to 4 times while it is inverted.
iii. With a new syringe for injection, withdraw approximately 0.6 mL from the HemaMax Drug Product vial.
iv. Replace the needle.
v. Gently expel extra liquid from the syringe until it reaches 0.5 mL mark.
vi. Label the syringe for injection with the subject information.

10 μg HemaMax (rHuIL12)

Drug Product Dose Preparation

Requires:

1 vial of HemaMax (rHuIL-12) Drug Product, 1 BD Syringes and 2 BD 25G ⅝ needles

i. Withdraw approximately 0.6 mL from the HemaMax Drug Product vial.

ii. Replace the needle.

iii. Gently expel extra liquid from the syringe until it reaches 0.5 mL mark.

iv. Label the syringe for injection with the subject information.

20 μg HemaMax (rHuIL12)

Drug Product Dose Preparation

Requires:

2 vials of HemaMax (rHuIL-12) Drug Product, 2 BD Syringes and 3 BD 25G ⅝ needles
i. Withdraw entire content (as much as you can) from the HemaMax (rHuIL-12) Drug Product vial.
ii. Transfer the entire amount to a second HemaMax (rHuIL-12) Drug Product vial.
ix. Mix solution via several inversions of the vial 3 to 4 times and then gently tap the bottom of the vial 3 to 4 times while it is inverted.
iii. With a new syringe for injection, withdraw approximately 1.1 mL from the second HemaMax (rHuIL-12) Drug Product vial.
iv. Replace the needle.
v. Gently expel extra liquid from the syringe until it reaches 1.0 mL mark.
vi. Label the syringe for injection with the subject information.

While this invention has been disclosed with reference to specific embodiments, it is apparent that other embodiments and variations of this invention can be devised by those skilled in the art without departing from the true spirit and scope of the invention. The appended claims include all such embodiments and equivalent variations.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of’, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for increasing survival in a subject with cancer, wherein the method comprises:
administering a therapeutically effective amount of a composition comprising IL-12 to the subject receiving a predetermined chemotherapy and/or chemoradiotherapy regimen, wherein the administration of IL-12 reduces the likelihood and/or severity of occurrence of at least two cytopenias inducible by chemotherapy and/or chemoradiotherapy;
whereby the need for subsequent adjustment of the chemotherapy and/or chemoradiotherapy dose regimen resulting from the induction of cytopenia is mitigated.

2. The method of claim 1, wherein the chemotherapy and/or chemoradiotherapy dose regimen adjustment includes modification of dose amount and frequency of administration, including dose fraction/dose titration and/or dose reduction.

3. The method of claim 1, wherein the administration of IL-12 induces anti-tumor responses.

4. The method of claim 1, wherein the combined modality of the IL-12 administration with chemotherapy and/or chemoradiotherapy results in an increase in the survival of the subject.

5. The method of claim 4, wherein the survival is progression-free survival.

6. The method of claim 4, wherein the survival is the overall survival of the subject.

7. The method of claim 1, wherein the cytopenias include lymphopenia.

8. The method of claim 1, wherein the cytopenias are selected from at least two of neutropenia, leucopenia, anemia, thrombocytopenia, and lymphopenia.

9. The method of claim 1, wherein the cytopenias include neutropenia.

10. The method of claim 7, wherein the lymphopenia includes at least one of the lymphocyte subsets comprising CD4+, CD8+, CD8+memory cells, and NK cells.

11. A method of treating or preventing a subject from chemotherapy-induced cytopenias following exposure of the subject to chemotherapeutic agents, the method comprising:
administering a dose of a therapeutically effective amount of a pharmaceutical composition comprising substantially isolated IL-12 to the subject, whereby chemotherapy-induced cytopenias are diminished.

12. The method of claim 11, wherein the chemotherapeutic agent is administered as a combined modality in combination with exposure to a dose of radiation.

13. The method of claim 11, wherein the effective dose of IL-12 is less than about 300 ng/kg.

14. The method of claim 11, wherein the effective dose of IL-12 is given in two or more doses of less than about 300 ng/kg for each dose.

15. The method of claim 11, wherein the one or more effective dose(s) of IL-12 is less than about 200 ng/kg.

16. The method of claim 11, wherein the one or more effective dose(s) of IL-12 is less than about 100 ng/kg.

17. The method of claim 11, wherein the one or more effective dose(s) of IL-I 2 are given before exposure to the chemotherapeutic agent.

18. The method of claim 11, wherein the one or more effective dose(s) of IL-12 are given before and after exposure to the chemotherapeutic agent.

19. The method of claim 11, wherein the one or more effective dose(s) of IL-12 are given after exposure to the chemotherapeutic agent.

20. The method according to claim 11, wherein the IL-12 is a rHuIL-12.

21. The method of claim 11, where the chemotherapeutic agent is a platinum-based chemotherapy.
